(19) United States
(12) Patent Application Publication
(10) Pub. No.: US 2026/0290546 A1
(43) Pub. Date: Sep. 24, 2026

(54) SYSTEM

(71) Applicant: SoftBank Group Corp., Tokyo (JP)

(72) Inventor: Toshiki MURATA, Tokyo (JP)

(73) Assignee: SoftBank Group Corp., Tokyo (JP)

(21) Appl. No.: 19/567,350

(22) Filed: Mar. 16, 2026

(30) Foreign Application Priority Data
Mar. 19, 2025 (JP) 2025-045169

(51) Int. Cl. G16H 20/60; G06F 40/10; G06F 40/205; G16H 40/67

(52) U.S. Cl. CPC G16H 20/60; G06F 40/10; G06F 40/205; G16H 40/67

(57) ABSTRACT

A system includes a processor that is configured to provide an interface for inputting physical information and dietary preferences of a user, convert input information into digital data, transmit the digital data via a network communication unit, receive the transmitted digital data, store the received digital data in a database, and store the received digital data in an information processing device, generate, based on the stored data, a prompt for instructing a generative AI model to generate a meal plan, and generate the meal plan by using the prompt, and output the generated meal plan to a display device of the user.

### CROSS-REFERENCE TO RELATED APPLICATION

This application is based on and claims priority under 35 USC 119 from Japanese Patent Application No. 2025-045169 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional dietary management and meal planning systems require users to manually search for recipes, evaluate nutritional balance, and repeatedly adjust meal plans according to changes in their physical condition and dietary preferences. Such systems often lack an integrated mechanism for collecting user physical information and dietary preferences as structured digital data, and for using that data to dynamically generate personalized meal plans with the aid of a generative AI model. As a result, users face a heavy cognitive and operational burden when they attempt to maintain nutritionally balanced diets tailored to their individual health conditions. Furthermore, conventional systems do not provide a closed feedback loop in which a user's health condition is monitored over time through implemented meal plans and in which meal plans are automatically adjusted based on changes in that health condition. Therefore, there is a need for a system that can seamlessly obtain user physical information and dietary preferences, generate prompts for a generative AI model to automatically create nutritionally appropriate meal plans, and continuously monitor and adjust those plans in response to the user's evolving health status.

### SUMMARY

In order to solve the above-described problems, the present invention provides a system comprising a processor, wherein the processor is configured to provide an interface for inputting physical information and dietary preferences of a user, convert input information into digital data, transmit the digital data via a network communication unit, receive the transmitted digital data, store the received digital data in a database, and store the received digital data in an information processing device. The processor is further configured to generate, based on the stored data, a prompt for instructing a generative AI model to generate a meal plan, to generate the meal plan by using the prompt, and to output the generated meal plan to a display device of the user. In one aspect, the processor is configured to generate a prompt for instructing the generative AI model to generate a meal plan that takes nutritional balance into account based on the physical information and the dietary preferences of the user, thereby enabling automatic creation of meal plans tailored to user-specific health conditions and taste preferences. In another aspect, the processor is configured to implement a feedback loop that generates a prompt for instructing the generative AI model to monitor a health condition of the user through the meal plan and to adjust the meal plan as needed, thereby enabling continuous optimization of the meal plan in accordance with changes in the user's health status.

The term “system” refers to an arrangement of hardware and software components including at least one processor, memory, storage, communication interfaces, and peripheral devices that collectively execute the functions described in the claims.

The term “processor” refers to any hardware device or combination of devices that executes instructions, including but not limited to a central processing unit (CPU), a graphics processing unit (GPU), a digital signal processor (DSP), a microcontroller, or a programmable logic device configured to perform the claimed operations.

The term “interface for inputting physical information and dietary preferences of a user” refers to any hardware and software combination that allows the user or another entity to enter or provide data representing the user's physical characteristics and dietary preferences, including graphical user interfaces, web forms, mobile application screens, touch panels, voice input interfaces, or application programming interfaces.

The term “physical information” refers to data related to the physiological or biometric characteristics of the user, including but not limited to age, gender, height, weight, body mass index, body composition, medical history, current health conditions, and vital signs.

The term “dietary preferences” refers to data indicating the user's likes, dislikes, restrictions, and patterns regarding food and meals, including preferred cuisines, favorite ingredients, disliked foods, allergies, religious or ethical dietary rules, and goals such as weight loss or muscle gain.

The term “digital data” refers to information represented in a machine-readable format, including structured or unstructured data encoded as numeric, textual, binary, or other digital representations suitable for processing, storage, and transmission by electronic devices.

The term “network communication unit” refers to any hardware and software component or module that enables data communication over a wired or wireless network, including network interface cards, wireless communication modules, modems, and associated communication protocols.

The term “database” refers to an organized collection of data stored in one or more storage devices, managed by database management software, and configured to allow insertion, retrieval, update, and deletion of data related to user information and meal plans.

The term “information processing device” refers to any electronic device or system that includes at least one processor and memory and that is capable of executing programs to process, store, and manage data, including servers, cloud computing platforms, and local computing devices.

The term “prompt” refers to a structured or semi-structured input instruction or message that is generated based on stored data and that is provided to a generative AI model to specify conditions, constraints, and objectives for generating a meal plan.

The term “generative AI model” refers to a machine learning model, such as a neural network or language model, that is configured to generate new content or data outputs, including meal plans or meal descriptions, in response to provided prompts.

The term “meal plan” refers to a set of one or more meals, dishes, or recipes, optionally associated with timing, frequency, and portion information, that is generated for the user and that is based on the user's physical information, dietary preferences, and nutritional considerations.

The term “display device of the user” refers to any device that presents visual information to the user, including but not limited to a smartphone display, tablet display, computer monitor, smart watch display, television screen, or other graphical display unit.

The term “nutritional balance” refers to a condition in which the intake of energy and nutrients such as proteins, fats, carbohydrates, vitamins, minerals, and dietary fiber is arranged within recommended ranges or target values appropriate to the user's physical information and health goals.

The term “feedback loop” refers to a process in which the system monitors the user's health condition in relation to implemented meal plans, uses the monitored results to generate new prompts, and causes the generative AI model to adjust subsequent meal plans based on the monitoring results.

The term “health condition of the user” refers to a state of the user's physical health, including measured or estimated indicators such as body weight, blood pressure, blood glucose, cholesterol levels, body composition, and other clinical or wellness parameters.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

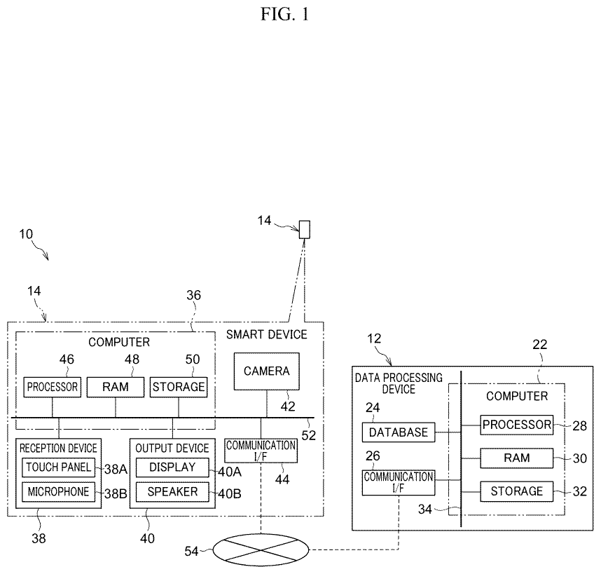

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

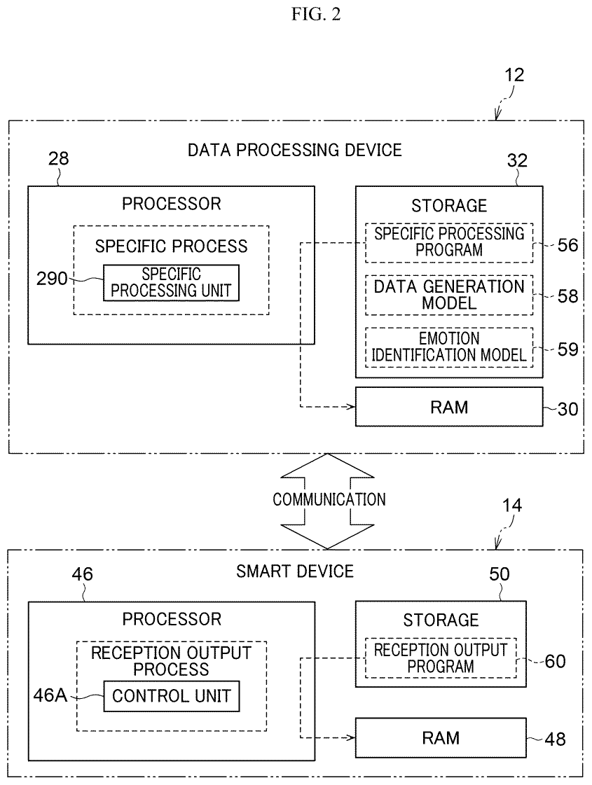

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented diet recommendation systems generally rely on simple rule engines or static recommendation logic that map a limited set of user attributes to pre-defined meal templates. Such systems typically treat health information and preference information as isolated inputs and do not tightly integrate heterogeneous data sources such as sensor-based activity data, longitudinal behavioral history, and free-form natural language feedback. As a result, existing systems often produce coarse-grained suggestions that fail to adapt to evolving user states, leading to low user engagement and poor personalization. From a computer technology standpoint, there are several concrete limitations. First, conventional architectures usually do not construct a unified machine-readable representation that combines health state indices, lifestyle patterns, and text-derived preference features into a coherent feature vector suitable for conditioning a generative model. This causes inefficiencies in processing pipelines, requires significant manual tuning of rules, and limits the ability of the system to utilize advances in generative artificial intelligence.

Second, existing systems commonly treat prompt sentences for generative models as static or manually authored text, disconnected from structured health and preference data. This separation prevents the computing system from programmatically generating prompts that accurately encode real-time user constraints and nutritional objectives. Such an approach leads to suboptimal utilization of computational resources, because generative models are invoked with incomplete or noisy context, degrading the accuracy and stability of generated meal plans.

Third, feedback from users is often stored as unstructured logs or simple ratings without being systematically processed through natural language processing and statistical analysis pipelines. This prevents the system from updating preference profiles and training data in a principled way and hinders the implementation of an effective feedback loop. Consequently, the underlying generative models are not efficiently retrained or fine-tuned based on user-specific performance signals, resulting in a static system that does not improve over time and that underutilizes computational learning capabilities.

Fourth, temporal evolution of user health states and responses to generated meal plans is rarely modeled as a state change process within the computing infrastructure. Without constructing a time-series state change model, conventional systems cannot dynamically adjust prompt contents and feature vectors according to health trends and behavioral patterns. This reduces the effectiveness of automatic monitoring and adaptive control of the recommendation process, leading to computational workflows that do not exploit historical context for optimization.

Accordingly, there is a need for an improved computer-implemented system that: (i) programmatically aggregates sensor-based and service-based health and lifestyle data into health state indices; (ii) applies natural language processing to text streams to construct and update preference profiles; (iii) automatically generates prompt sentences and numerical feature vectors that jointly condition a generative model; and (iv) implements a closed-loop architecture in which evaluation information and time-series records are used to update preference profiles, training data, and model parameters. Such a system should enhance the technical functioning of the recommendation engine by improving data representation, model conditioning, learning efficiency, and stability and relevance of the generated meal plans, thereby providing a concrete improvement to computer technology in the field of personalized content generation.

The specific processing by the specific processing unit 290 of the data processing device 12 in Example 1 is realized by the following means.

The present invention provides a server comprising at least one processor and at least one storage device storing instructions that, when executed by the at least one processor, cause the server to provide an acquisition interface for receiving bodily condition information, lifestyle information, and meal preference information from a user terminal; to convert the received information into digital data and store the digital data in an information processing apparatus; to acquire biophysical activity information and behavioral history information from external measurement apparatuses and external information services and aggregate the acquired information into a health state index; to execute natural language processing on text information from the user and the external information services to extract feature information relating to meal preferences, prohibitions, and social situations and to generate a preference profile; to convert the health state index and the preference profile into a numerical feature vector and automatically generate a prompt sentence summarizing the numerical feature vector and constraint conditions of the user; to supply the prompt sentence and the numerical feature vector as conditioning inputs to a generative model to generate meal planning information; to structure the generated meal planning information into meal proposal information including date, intake time, dish, and nutrition attributes and transmit the meal proposal information to the user terminal; to receive evaluation information and additional text information from the user terminal regarding the meal proposal information; to perform natural language processing and statistical processing on the received evaluation information and additional text information to update the preference profile and training information for the generative model; and to perform, based on the updated preference profile and training information, a learning or fine-tuning process of the generative model and further update the prompt sentence and numerical feature vector generation so as to form a feedback loop that dynamically adapts meal planning information to temporal changes in the user's health state and behavior. This enables the computing system to implement an integrated, machine-optimized pipeline that unifies heterogeneous sensor data and natural language data into structured conditioning inputs for a generative model, programmatically generates context-rich prompt sentences, and continuously refines model parameters and user representations based on time-series feedback, thereby improving the technical performance, adaptability, and accuracy of computer-implemented meal plan generation.

The term “bodily condition information” refers to information indicating a physical state of a user, including at least one of biometric measurements, physiological parameters, body composition indicators, and medical status indicators obtained from sensors, questionnaires, or external data sources.

The term “lifestyle information” refers to information indicating daily habits and behavior patterns of a user, including at least one of activity levels, sleep patterns, work schedules, eating schedules, and social interaction patterns.

The term “meal preference information” refers to information indicating likes, dislikes, tendencies, or constraints of a user with respect to food and meals, including at least one of preferred ingredients, disliked ingredients, cuisine types, flavor profiles, and portion size preferences.

The term “acquisition interface” refers to a hardware or software component configured to receive information from a user or an external system, including at least one of a graphical user interface, an application programming interface, a sensor interface, and a communication interface.

The term “digital information” refers to information represented in a machine-readable format, including at least one of numerical values, character strings, structured records, and encoded signals suitable for processing by a computing device.

The term “information processing apparatus” refers to a computing entity including at least one processor and at least one storage device, configured to execute instructions for data acquisition, data transformation, analysis, and generation of results.

The term “communication unit” refers to a component configured to transmit and receive information between computing entities, including at least one of wired network interfaces, wireless network interfaces, and standardized communication protocols.

The term “external measurement apparatus” refers to an external device configured to measure physical or physiological parameters of a user, including at least one of a wearable sensor, a health monitoring device, and an environmental sensor.

The term “external information service” refers to a network-accessible service that provides data relating to a user, including at least one of a health data service, a social networking service, and a cloud-based activity tracking service.

The term “biophysical activity information” refers to information indicating physical activities or physiological activity levels of a user over time, including at least one of step counts, heart rate data, energy expenditure, and exercise logs.

The term “behavioral history information” refers to information indicating past behavior of a user in a time-series manner, including at least one of meal logs, activity logs, social interactions, and application usage records.

The term “health state index” refers to a computed indicator representing a health-related state of a user, derived from at least one of biophysical activity information, behavioral history information, bodily condition information, and lifestyle information.

The term “text information” refers to information expressed in a natural language format, including at least one of short messages, feedback comments, social media posts, and free-form descriptions.

The term “natural language processing” refers to processing performed by a computing device on text information to derive structured information, including at least one of tokenization, part-of-speech tagging, entity recognition, keyword extraction, sentiment analysis, and dependency analysis.

The term “feature information” refers to structured attributes extracted from raw data, including at least one of numerical features, categorical features, and symbolic descriptors suitable for input to a computational model.

The term “preference profile” refers to a data structure representing inferred or declared preferences, constraints, and tendencies of a user, consolidated from at least one of meal preference information, feedback information, and text information.

The term “numerical feature vector” refers to a sequence or array of numerical values representing one or more attributes of a user, including at least health state indices, preference profile values, and constraint parameters, in a form suitable for numerical computation by a model.

The term “constraint conditions” refers to conditions that restrict or guide generation of meal planning information, including at least one of dietary restrictions, nutritional targets, timing constraints, and social context constraints.

The term “prompt sentence” refers to a natural language or structured text sequence generated by a computing device to describe context, constraints, and objectives, and supplied as an input condition to a generative model.

The term “generative information processing model” refers to a computational model configured to generate output information based on input conditions, including at least one of a probabilistic model, a neural network model, and a sequence generation model.

The term “meal planning information” refers to information representing one or more recommended meals over a time period, including at least one of meal compositions, meal timings, and associated nutritional attributes.

The term “meal proposal information” refers to a structured representation of meal planning information, including at least date information, intake time information, dish information, and nutrition information, stored in a format accessible to a user terminal.

The term “dish information” refers to information describing a meal item, including at least one of a dish name, ingredient list, portion size, and preparation description.

The term “nutrition information” refers to information indicating nutritional properties of a meal or dish, including at least one of energy value, macronutrient composition, micronutrient content, and dietary fiber content.

The term “terminal apparatus” refers to a user-operated device that communicates with a server, including at least one of a portable communication terminal, a desktop computing device, and a display-equipped embedded device.

The term “display unit” refers to a component configured to visually present information to a user, including at least one of a liquid crystal display, an organic light-emitting display, and a graphical user interface.

The term “evaluation information” refers to information indicating a user's reaction or assessment of presented meal proposal information, including at least one of ratings, selections, categorical feedback, and interaction logs.

The term “additional text information” refers to free-form or semi-structured text provided by a user in association with evaluation information, including at least one of comments, requests, and explanations.

The term “statistical processing” refers to processing performed by a computing device to summarize or infer patterns from data, including at least one of aggregation, correlation analysis, distribution estimation, and parameter estimation.

The term “training information” refers to information used to adjust parameters of a generative information processing model, including at least one of training data pairs, labels, reward signals, and feedback-derived metrics.

The term “learning process” refers to a process in which a computing device adjusts parameters of a model based on training information so as to reduce an error metric or improve a performance metric.

The term “fine-tuning process” refers to a learning process in which a pre-trained model is further trained on additional data, including user-specific or domain-specific data, to adapt the model to a particular task or user group.

The term “feedback loop” refers to an arrangement in which outputs of a system, together with user evaluation and temporal data, are fed back into the system to update internal states, models, or parameters so as to influence subsequent outputs.

In one embodiment, a server, a terminal, and a user cooperate to implement the invention. The server includes at least one processor, at least one memory, and at least one network interface, and is realized, for example, on a general-purpose computation platform such as a rack-mounted computer, a virtual machine instance, or a containerized environment running on a cloud computing infrastructure. The server executes an operating system such as a general-purpose server operating system and executes application software implemented, for example, using a programming language such as a general-purpose high-level language. The terminal is realized by a user-operated device such as a smartphone, a tablet computer, a portable computer, or a stationary computer, executing a client application. The terminal includes at least a display unit, an input unit such as a touch panel or keyboard, a network interface, and a local storage device.

The server stores, in the memory, multiple software modules including at least a data acquisition module, a data normalization module, a natural language processing module, a feature construction module, a prompt generation module, a generative AI model inference module, a meal plan structuring module, a feedback analysis module, a model training module, and a communication module. The server stores, in a database system, multiple data structures including at least a user profile table, a health state index table, a preference profile table, a meal plan table, a feedback table, and a model configuration table.

The server uses, in one embodiment, a relational database management system to store user-related records. The user profile table stores fields such as a user identifier, demographic attributes, declared dietary restrictions, and device linkage information. The health state index table stores, for each user and for each time segment, aggregated indicators such as total steps, average heart rate, sleep duration, and other computed statistics. The preference profile table stores numerical values and flags that represent extracted preferences and constraints, such as a low-carbohydrate preference score, a dairy-free flag, and a late-evening-meal avoidance score. The meal plan table stores generated meal planning information, including for each day and meal: dish identifiers, textual descriptions, and nutritional values. The feedback table stores evaluation information such as ratings, categorical tags, and free-text comments associated with specific meal items and meal plans.

The server acquires bodily condition information and lifestyle information from external measurement apparatuses and external information services. For example, the server communicates with a wearable device platform API or a health tracking service API using the communication module. The server transmits authenticated HTTP requests containing access tokens and receives responses containing time-stamped measurements such as step counts, heart rate samples, and sleep intervals. The server parses the responses and writes normalized records into an intermediate table representing raw sensor readings. The server then executes aggregation operations, for example using a data processing library or database aggregation queries, to compute daily or hourly summaries and to derive the health state indices. The server stores these derived indices in the health state index table.

The server acquires text information that contains user feedback and social-context data. The terminal displays input screens that allow the user to enter free-text comments such as “I want to cut down on sugar” or “Please avoid heavy meals late at night,” and to select structured options such as “too salty” or “portion too large.” The terminal transmits these data items to the server via a network interface, using secure communication protocols. The server stores the raw text in the feedback table along with metadata such as timestamp and identifiers of the corresponding meal items. Additionally, the server may acquire text information from external information services such as social communication platforms, based on the user's authorization. The server filters such text by detecting food-related keywords using pattern matching, and stores the filtered text as part of the text information dataset.

The server performs natural language processing on the stored text information. The natural language processing module loads a language model and processing pipeline that perform tokenization, part-of-speech tagging, dependency parsing, and named entity recognition. The server processes each text record to identify phrases associated with dietary preferences, prohibitions, and social contexts. For example, the server identifies phrases such as “cut down on carbs” as indicating a low-carbohydrate preference, “no dairy” as indicating a dairy exclusion constraint, and “dinner with colleagues” as indicating a recurring social-meal context. The server encodes these phrases as feature information by mapping them to predefined dimensions in the preference profile, for example, incrementing numerical scores or setting boolean flags.

The server constructs and updates the preference profile for each user based on the aggregated feature information. The preference profile includes, in one embodiment, a vector of continuous values in a fixed-dimensional space, where each dimension represents a strength of preference or constraint. The server applies statistical processing, such as exponential moving averaging or weighted updates, to reflect both recent and historical text information. By doing so, the server mitigates noise and short-term fluctuations in user feedback, leading to more stable and accurate preference representations and thereby improving subsequent model conditioning and inference accuracy.

The server constructs a numerical feature vector that combines the health state index and the preference profile. In one embodiment, the server normalizes numerical dimensions such as steps, sleep duration, and heart rate using a normalization method such as min-max scaling or z-score normalization. The server also encodes categorical items such as dietary type or activity level category using one-hot encoding or embedding indices. The server concatenates the normalized health-related features and the preference-related features to form a numerical feature vector suitable for input into a machine learning model.

The server generates a prompt sentence to condition a generative AI model. The prompt generation module assembles human-readable text segments that summarize the numerical feature vector and the user's constraint conditions. For example, the server generates a prompt sentence such as:

“The user has an average of 8,000 steps per day, sleeps about 6 hours per night, and wants to reduce carbohydrates and sugar. The user prefers light dinners and often eats lunch with colleagues. Please generate a 3-day low-carb meal plan that is suitable for social lunches and supports gradual weight loss.”

In another example, the server generates a prompt sentence such as:
“Based on the user's recent feedback that they want to cut down on sugar and prefer lighter dinners, and considering their activity data showing low activity and short sleep, please propose a one-week meal plan with reduced sugar and light evening meals, including brief nutritional information for each meal.”

The server, by automatically composing such prompt sentences from structured health indices and preference profiles, avoids relying on static, manually authored prompts. This dynamic prompt generation results in more precise and context-rich conditioning of the generative AI model. As a consequence, the model output has higher relevance and consistency, reducing the need for manual post-processing and improving the overall computational efficiency of the system.

The server executes a generative AI model inference process based on the prompt sentence and the numerical feature vector. In one embodiment, the generative AI model is a neural network having a transformer-based architecture with multiple self-attention layers, feed-forward layers, and layer normalization components. The model parameters are stored in the memory and loaded into computation units such as central processing units or graphics processing units. The model receives as input tokenized representations of the prompt sentence and one or more auxiliary inputs representing the numerical feature vector, for example concatenated to special control tokens or injected via additional conditioning layers. The server defines, for the generative AI model, hyperparameters including the number of layers, the number of attention heads, the dimensionality of hidden states, and the vocabulary size. The server also defines an output format in which the model generates sequences describing meal items, meal times, and brief nutritional comments. For example, the model may generate text of the following form:
“Day 1: Breakfast-scrambled eggs with vegetables; Lunch-grilled chicken salad; Dinner-tofu and vegetable stir-fry without rice. Day 2: . . . ”

The server performs inference by invoking the generative AI model with a decoding method such as greedy decoding, beam search, or sampling-based decoding (e.g., top-k or nucleus sampling). The server limits the maximum output length and may enforce certain output patterns using constrained decoding rules. These rules, for example, restrict the appearance of ingredients that are incompatible with the user's dietary constraints, based on a mask that is precomputed from the preference profile and health state index.

The server structures the raw generated text into machine-usable meal planning information. The meal plan structuring module parses the generated text by identifying delimiters such as day labels and meal labels, and by matching known patterns for dishes and times. The server extracts dish names, associates them with meal slots (such as breakfast, lunch, and dinner), and assigns dates or relative days. The server may consult an ingredient database and a nutritional database to derive nutrition information for each dish through look-up and summation operations. The server then writes structured records into the meal plan table, associating each dish with nutritional attributes such as estimated energy, macronutrient breakdown, and key micronutrients.

The terminal receives the meal proposal information from the server. The terminal, using the communication module, sends a request message including the user identifier and authentication information to the server. The server responds with structured meal proposal information. The terminal parses the received information and renders a graphical user interface that lists days and meal items. The terminal allows the user to scroll through days, tap on individual dishes to view details, and view indicators such as “low carb,” “high protein,” or “light dinner.” The terminal thereby provides an intuitive interface for consuming the generated meal plans.

The user interacts with the meal plan and provides evaluation information and additional text information. The terminal displays input widgets such as rating controls, toggle buttons for “like” or “dislike,” and text input fields. The user can, for example, rate a specific dinner as “too heavy,” or write a comment such as “Please avoid rice for dinner.” The terminal packages these interaction data as a structured payload, including identifiers for the associated meal items, and transmits the payload to the server.

The server analyzes the feedback using the feedback analysis module. The server performs natural language processing on the free-text component to extract qualifiers such as “too heavy” and “avoid rice.” The server maps such phrases to adjustments in the preference profile, for example by increasing the weight of a “light dinner” dimension and adding a constraint to reduce grain-based carbohydrates in the evening meals. The server also interprets numerical ratings as reward or penalty signals that indicate how well the generated meals align with the user's preferences and health objectives.

The server uses the updated preference profile and accumulated feedback as training information for the generative AI model. The model training module selects pairs of conditioning inputs (prompt sentences and numerical feature vectors) and desired outputs (meal plans that received positive feedback or improved over time). The server defines a loss function that combines a language modeling loss (for example, cross-entropy between predicted tokens and target tokens) with an auxiliary loss derived from feedback signals (for example, a penalty term for patterns correlated with negative feedback). The server performs optimization using an algorithm such as stochastic gradient descent or an adaptive variant, and updates the model parameters in the memory. The server may perform this training process on a separate training server that has enhanced computation resources, and then deploy the updated model to the inference server.

The server's integration of heterogeneous sensor data, behavioral data, and text-based feedback into a single modeling pipeline produces several technical effects. The conversion of these data into a unified numerical feature vector reduces the dimensionality and redundancy of the input, increasing computational efficiency during model inference and training. The automatic generation of context-rich prompt sentences based on structured indices enables the generative AI model to operate under more precise constraints and objectives, thereby improving output accuracy. The feedback loop, implemented through systematic natural language processing and statistical updating of preference profiles, allows the system to adapt model parameters over time according to actual performance signals, thus reducing prediction error and increasing personalization quality.

The server, by modeling temporal evolution of the health state index, the meal planning information, and the evaluation information, constructs a state change model for each user. The server utilizes this model to adjust prompt sentences and numerical feature vectors in a time-aware manner, for example by emphasizing certain constraints after detecting adverse trends in health indicators or repeated negative feedback. This temporal adaptation contributes to technical improvements in both stability and responsiveness of the recommendation process, as the generative AI model is conditioned not only on instantaneous states but also on trends and patterns.

In another embodiment, the generative AI model is combined with a rule-based post-processing module that enforces non-conventional constraints that are difficult to express in simple human rules. For instance, the server applies rules that detect conflicts between model-generated dishes and long-term health objectives, such as limiting the frequency of high-sugar desserts or enforcing minimum vegetable servings across a weekly plan. These hybrid mechanisms exploit the strengths of learned generative behavior while ensuring compliance with domain-specific safety criteria, resulting in more reliable outputs and reduced need for human correction.

The system, thus, does not merely automate human meal planning decisions. Instead, the server implements a specific architecture, data structures, and learning procedures that allow the computer system to process large-scale, heterogeneous data streams in ways that are not feasible manually. The server improves processing speed by leveraging batch operations on sensor data and parallelized neural network inference. The server improves accuracy by tightly coupling structured health indices with text-derived preference features to condition the generative AI model. The server improves data management by maintaining normalized tables and stateful preference profiles that can be efficiently queried and updated. The server also reduces communication load by transmitting compact structured representations and by performing most computationally intensive operations centrally, rather than repeatedly exchanging raw data with the terminal.

In yet another embodiment, different generative model architectures can be used. The server may employ a sequence-to-sequence encoder-decoder network with attention mechanisms, in which the encoder processes the prompt sentence and auxiliary conditioning vectors, and the decoder generates the meal plan text. Alternatively, the server may utilize a unified transformer model with special tokens marking the start and end of each day and meal section. The server may also adjust the model's architecture parameters, such as layer count and hidden dimension size, based on available computation resources and desired response latency. These architectural variations are all encompassed within the scope of the invention, as they share the essential mechanism of conditioning a generative model on health state indices, preference profiles, and dynamically generated prompt sentences.

Similarly, the natural language processing methods used by the server can be varied. In some embodiments, a rule-based pattern matcher and keyword extractor may be used to identify specific phrases, while in other embodiments, a trained classifier or sequence labeling model may be used to categorize text segments into preference-related classes. Different languages and models can be employed according to the locale of the user. Regardless of the specific implementation, the core function of transforming unstructured text into structured feature information that populates the preference profile remains consistent.

The terminal can also vary across embodiments. In some cases, the terminal is a dedicated application on a mobile device. In other cases, the terminal is a browser-based client or an embedded interface in a kitchen appliance. The role of the terminal, namely to present meal proposal information, to acquire user input and feedback, and to relay these to the server, is maintained.

Through these embodiments and variations, the system provides a concrete implementation that enables others skilled in the art to practice the invention. The server's specific data structures, algorithmic flows, and generative AI model conditioning strategies work together to achieve technical improvements in the field of personalized meal planning and, more broadly, in computer-implemented generative recommendation technologies.

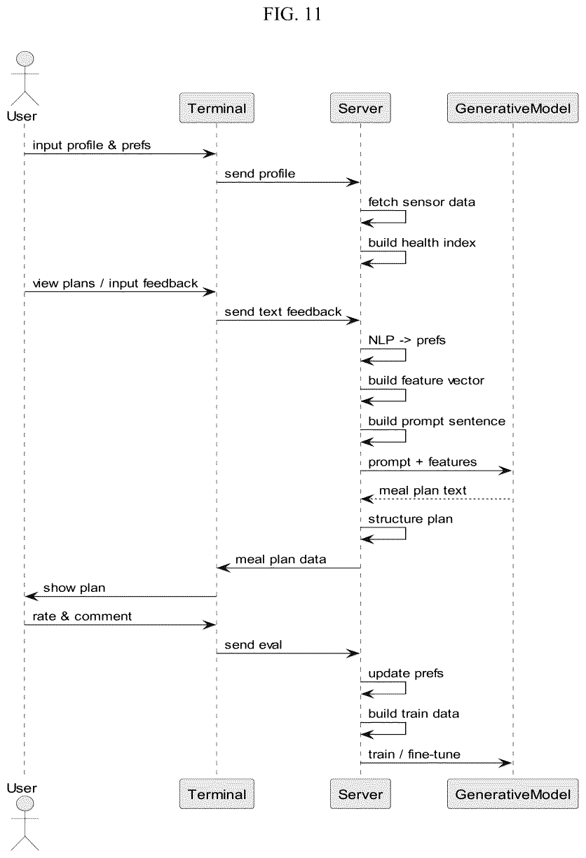

The following describes the processing flow using FIG. 11.

### Step 1

The terminal presents a registration and consent interface to the user.

The user inputs profile data such as age, height, weight, basic dietary restrictions, and authorizes linkage to external measurement apparatuses and external information services. The terminal uses the input as an input dataset, packages the profile data and authorization tokens into a structured message, and transmits the message to the server via a secure communication channel.

The server receives the message as input, validates formats and permissions, and writes normalized user profile records and linkage records into a user profile table as output.

### Step 2

The server acquires bodily condition information and lifestyle information from external measurement apparatuses and external information services.

The server uses stored access tokens and user linkage records as input, sends authenticated requests to external APIs, and receives raw sensor data such as time-stamped steps, heart rate signals, and sleep intervals as output.

The server parses the raw sensor data, converts units and time zones, and stores the normalized raw readings into an intermediate sensor reading table.

The server then aggregates these readings per user and time segment, calculating daily totals and averages to produce aggregated health metrics as output.

### Step 3

The server generates a health state index from the aggregated health metrics.

The server uses the aggregated health metrics (e.g., total steps, average resting heart rate, sleep duration) as input, applies statistical operations such as normalization and weighted averaging, and computes composite indicators such as activity level scores and sleep quality scores.

The server combines these indicators into a vector representing the health state index and stores the vector in a health state index table as output.

### Step 4

The terminal presents a feedback and comment interface to the user.

The user reviews displayed meal plans or enters general dietary intentions such as “I want to reduce sugar” or “Please avoid heavy meals at night.”

The terminal uses the user's ratings, checkbox selections, and free-text comments as input, groups them into a feedback object, and includes references to relevant meal items or general preference categories.

The terminal sends this feedback object to the server as output via a network request.

### Step 5

The server acquires additional text information from external information services if authorized.

The server uses stored external account tokens and query parameters as input, calls external text-based services (such as social communication feeds) and retrieves posts or entries mentioning food, diet, or health as output.

The server filters the retrieved text by applying keyword searches and basic pattern matching, discarding unrelated entries, and stores the relevant text segments into a text information table.

### Step 6

The server performs natural language processing on stored text information.

The server uses the text entries from the feedback table and text information table as input, loads a natural language processing pipeline, and applies tokenization, part-of-speech tagging, and phrase extraction.

The server detects expressions related to meal preferences, prohibitions, and social contexts, such as “cut down on carbs,” “no dairy,” or “dinner with colleagues,” and converts these expressions into structured feature information as output, for example preference scores or flags.

The server writes these features into an intermediate feature table.

### Step 7

The server constructs and updates a preference profile for each user.

The server uses the feature information from the intermediate feature table and existing preference profile values as input, applies update rules such as weighted averaging, decay factors, or thresholds, and calculates updated scores and flags for each preference dimension. The server produces an updated preference profile vector as output and stores it in the preference profile table associated with the user.

### Step 8

The server constructs a numerical feature vector that combines health and preference information.

The server uses the health state index vector and the preference profile vector as input, normalizes continuous dimensions (e.g., steps, BMI) using scaling functions, and encodes categorical attributes via one-hot encoding or index mapping.

The server concatenates these processed values into a single numerical feature vector as output, suitable for conditioning a generative AI model.

### Step 9

The server generates a prompt sentence for the generative AI model.

The server uses the numerical feature vector and constraint conditions from the user profile and preference profile as input, converts numerical values into human-readable phrases (e.g., “average of 8,000 steps per day,” “sleeps about 6 hours”), and retrieves textual templates for common constraints (e.g., “wants to reduce carbohydrates and sugar,” “prefers light dinners”).

The server assembles these phrases into a coherent prompt sentence as output, such as: “The user has an average of 8,000 steps per day, sleeps about 6 hours per night, and wants to reduce carbohydrates and sugar. The user prefers light dinners and often eats lunch with colleagues. Please generate a 3-day low-carb meal plan that is suitable for social lunches and supports gradual weight loss.”

### Step 10

The server performs inference using the generative AI model.

The server uses the prompt sentence and the numerical feature vector as input, tokenizes the prompt sentence into model tokens and encodes the numerical vector as auxiliary conditioning input, and passes both into a generative AI model with a defined neural network architecture.

The server executes the model's forward pass with decoding parameters (e.g., beam width or sampling thresholds) and generates a text sequence describing meal planning information as output, such as day-by-day meal descriptions with breakfast, lunch, and dinner entries.

### Step 11

The server structures the generated text into meal proposal information.

The server uses the generated text sequence from the generative AI model as input, parses the sequence by detecting day markers, meal markers, and dish descriptions, and segments the text into structured meal entries.

The server optionally queries an ingredient and nutrition database using the dish names as input, computes nutritional summaries, and associates each meal with nutrition information. The server stores the resulting structured records, including date, intake time, dish information, and nutrition information, as meal proposal information in the meal plan table as output.

### Step 12

The terminal requests and receives meal proposal information from the server.

The terminal uses the user identifier and authentication token as input, sends a request to the server for the latest meal proposal.

The server uses the request as input, queries the meal plan table for the most recent valid meal proposal for the user, and returns the structured meal proposal information as output. The terminal receives the meal proposal information and passes it to the display module.

### Step 13

The terminal presents the meal proposal information to the user.

The terminal uses the structured meal proposal information as input, formats it into a graphical layout, and renders lists or calendar views grouping meals by day and time on the display.

The terminal displays key details such as dish names, brief descriptions, and nutritional tags as output on the screen so that the user can review proposed meals.

### Step 14

The user reviews the presented meal proposal and provides evaluation information.

The user uses the terminal interface as input medium, selects options such as “like” or “dislike,” adjusts rating scales, and enters comments such as “too heavy at night” or “please avoid rice at dinner.”

The terminal captures these interactions as evaluation information and additional text information as output and associates them with specific meal items or days.

### Step 15

The terminal transmits the evaluation information and additional text information to the server.

The terminal uses the user's feedback, meal identifiers, and timestamps as input, constructs a feedback payload, and sends it via a secure communication channel to the server.

The server receives the feedback payload as output of this step and writes it into the feedback table.

### Step 16

The server analyzes evaluation information and updates the preference profile.

The server uses the evaluation information (ratings, tags) and additional text information as input, applies natural language processing to extract new preference cues (e.g., “too heavy,” “avoid rice at dinner”), and uses statistical rules to translate ratings into reward or penalty values.

The server updates preference dimensions such as “light dinner preference” or “evening carbohydrate constraint” in the preference profile, producing an updated preference profile as output that reflects the user's current tastes and constraints.

### Step 17

The server prepares training data for updating the generative AI model.

The server uses historical pairs of numerical feature vectors, prompt sentences, generated meal plans, and associated feedback signals as input, selects examples according to criteria such as recency or diversity, and labels them with target outputs or reward scores. The server organizes these pairs into a training dataset as output, including tokenized input sequences, target token sequences, and auxiliary feedback-based labels.

### Step 18

The server performs learning or fine-tuning of the generative AI model.

The server uses the training dataset as input, defines a loss function combining language modeling loss and feedback-based penalty or reward components, and executes an optimization algorithm to adjust model parameters.

The server updates the weights of the neural network, saves a new model checkpoint as output, and optionally deploys the updated model to the inference environment, so that subsequent inference calls use the refined parameters.

### Step 19

The server reconstructs prompt sentences and numerical feature vectors based on updated states.

The server uses the latest health state indices, updated preference profiles, and state change models as input, recalculates normalized numerical feature vectors, and regenerates prompt sentences that emphasize newly important constraints or changed trends.

The server outputs refreshed conditioning inputs that will be used in the next inference cycle, thereby closing the feedback loop and enabling continuous adaptation of meal planning information.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computer-implemented meal recommendation systems generally suffer from several technical limitations. First, such systems typically treat user preference data, biometric data, and delivery service data as separate information streams and process them in isolation. As a result, servers frequently execute fragmented workflows in which health data is processed in one subsystem, social or preference data is processed in another subsystem, and delivery ordering is handled by yet another subsystem, with only minimal coordination. This fragmented architecture leads to inefficient use of processing resources, repeated data transformations, and increased latency across network interfaces.

Second, conventional systems usually rely on static rule sets or simple parameterized queries to generate recommendations. In such systems, a processor often executes fixed decision trees or hand-crafted filtering logic over databases. This approach is not well suited for integrating heterogeneous data sources such as biometric time-series data, unstructured text representing social posts, and external menu catalogs from delivery platforms. The processor must perform multiple ad hoc conversions and mappings, often leading to high computational overhead, redundant data parsing, and increased error rates when correlating items across systems.

Third, in a typical architecture, the interaction between a recommendation engine and an external delivery platform is weakly coupled. Even when a machine learning model outputs a meal plan, a separate mapping procedure must be manually designed to select actual items from a delivery catalog. This mapping is frequently implemented as a heuristic search or keyword matching routine that runs independently of the recommendation logic. As a consequence, the system cannot reuse or adapt the internal knowledge of the recommendation engine to optimize both the recommendation and the item selection, leading to additional processing stages, inconsistent results, and degraded responsiveness.

Fourth, feedback handling is often limited to simple logging, where user ratings or comments are stored but not immediately reflected in the recommendation pipeline in a structured manner. Servers typically require offline retraining of models or manual adjustment of rules, which means that the running system cannot promptly adapt its internal data representations or model inputs. This restricts the system's ability to form an efficient, closed feedback loop that dynamically reconfigures the recommendation behavior based on fresh feedback signals, thereby limiting overall system performance and user responsiveness.

These limitations manifest as concrete computer-technical problems: inefficient dataflows across components; repeated parsing and reformatting of heterogeneous data; latency and resource overhead caused by separate engines for recommendation, mapping, and feedback integration; and difficulties in maintaining consistency of user-specific state across these engines. There is a need for a computer-implemented system and server-side processing architecture that tightly integrates (i) collection and normalization of biometric and behavioral information, (ii) natural-language-based extraction of food-related preferences and social context, (iii) generation and use of structured prompt sentences for a generative AI model, (iv) AI-assisted mapping between abstract meal plans and concrete delivery menu items, and (v) feedback-based updating of user profiles, in a unified, looped workflow. Such an architecture should improve computational efficiency, reduce redundant processing and network round-trips, and enhance the adaptability and accuracy of recommendations by using a single generative AI model as a core reasoning component for planning, mapping, and feedback integration.

The specific processing by the specific processing unit 290 of the data processing device 12 in Application Example 1 is realized by the following means.

The present invention provides a server comprising a processor configured to receive biometric information and behavioral information of a user from a terminal via a communication network, standardize the biometric information and the behavioral information, and store the standardized biometric information and behavioral information as a user profile; analyze text information including posting information of the user obtained via an external information providing device by using a natural language processing function, extract preference information relating to meals and social situation information from the text information, and store the preference information and the social situation information in association with the user profile; generate, based on the user profile, the preference information, and the social situation information, a prompt sentence to be input to a generative AI model, supply the prompt sentence to the generative AI model, and cause the generative AI model to generate a meal plan adapted to a health condition and a living situation of the user and reflecting the preference information and the social situation information; output the generated meal plan to a display unit of the terminal and accept selection input of the meal plan by the user via the terminal; acquire candidate menu information provided from an information providing device of an external delivery service device in order to obtain an actually provided food corresponding to the meal plan selected by the user, generate a further prompt sentence for the generative AI model based on the candidate menu information and the meal plan, and, by using the further prompt sentence, specify, from among the candidate menu information, the actually provided food that most closely matches the meal plan; transmit order information including the specified actually provided food to the external delivery service device, acquire progress information relating to a delivery state based on the order information from the external delivery service device, and notify the progress information to the terminal; and acquire evaluation information from the user regarding the actually provided food, update the preference information based on the evaluation information, generate a new prompt sentence reflecting the updated preference information, and supply the new prompt sentence to the generative AI model as a condition for generation of a subsequent meal plan. This enables integrated and iterative server-side processing in which heterogeneous data sources are normalized into a unified user profile, a generative AI model is centrally driven by structured prompt sentences for both meal planning and menu-item selection, and user feedback is immediately incorporated into subsequent prompt generation, thereby reducing redundant data transformations, shortening processing paths between recommendation and ordering, improving utilization of computational resources, and enhancing the responsiveness and accuracy of computer-implemented meal recommendation and delivery coordination.

The term “processor” refers to a hardware logic element or a combination of hardware and software resources, such as a central processing unit, a graphics processing unit, or a microcontroller, that executes instructions to perform data processing operations described in the present specification.

The term “terminal” refers to an electronic device operated by a user, such as a portable information processing device or a wearable information processing device, that is configured to collect information from the user, transmit the information to a server, receive information from the server, and present the received information to the user.

The term “biometric information” refers to information representing a physical or physiological state of a user, including, for example, heart rate, body weight, body temperature, sleep duration, and physical activity amount, that is measurable by a sensor and processable by an information processing device.

The term “behavioral information” refers to information representing actions, habits, or lifestyle patterns of a user, including, for example, movement amount, activity logs, time-of-day usage patterns, and device interaction history, that is detectable by a sensor or an application and processable by an information processing device.

The term “user profile” refers to structured data stored in a storage device, the structured data including at least biometric information and behavioral information associated with a particular user and optionally including derived indicators, trends, or classifications obtained by processing such information.

The term “storage device” refers to a physical or virtual data retention component, such as a memory device, a magnetic storage device, a solid state storage device, or a database system, that is configured to store digital data used by the processor.

The term “communication network” refers to a wired or wireless communication infrastructure, such as a local area network, a wide area network, or a cellular network, that enables transmission and reception of digital data between the terminal, the server, and external devices.

The term “external information providing device” refers to an information processing device or service, such as a social networking service platform or a content distribution service, that provides text information or other digital content relating to a user to the server via the communication network.

The term “text information” refers to information expressed in a natural language character sequence, including, for example, messages, postings, comments, and descriptions, that is processable by natural language processing algorithms.

The term “posting information” refers to text information that a user has published or transmitted via an external information providing device, and that can be acquired by the server through an application programming interface or other communication mechanism.

The term “natural language processing function” refers to a software-implemented procedure or model that processes text information expressed in human language to perform operations such as tokenization, part-of-speech tagging, entity extraction, sentiment analysis, or intent detection.

The term “preference information” refers to information indicating tendencies of liking or disliking by a user with respect to items, categories, or attributes related to meals, such as favored ingredients, cuisines, cooking methods, or dietary styles, derived from analysis of text information or feedback data.

The term “social situation information” refers to information representing a user's social context or interpersonal circumstances related to eating behavior, including, for example, eating with others, social events, and frequency or timing of group meals, derived from analysis of text information or other data sources.

The term “generative AI model” refers to a machine-learned model that, in response to an input including a prompt sentence, is configured to generate an output including, for example, text, structured data, or recommendations, the machine-learned model being trained using example data and implemented on a computing apparatus.

The term “prompt sentence” refers to a structured input expression, represented in a natural language or in a combination of natural language and formal notation, that specifies conditions, constraints, or context for the generative AI model to generate an output such as a meal plan or a selection of menu items.

The term “meal plan” refers to digital data representing one or more proposed food intakes for a user, including at least information about a dish or menu item and optionally information concerning ingredients, nutritional content, energy amount, or timing of intake. The term “display unit” refers to a component of a terminal, such as a liquid crystal display or an organic light-emitting diode display, that is configured to present visual information, including meal plans and progress information, to the user.

The term “candidate menu information” refers to digital data representing a plurality of food items or menu entries that are available for provision to the user via an external delivery service, the digital data including at least an identification of each food item and optionally information concerning composition, description, or price.

The term “external delivery service device” refers to an information processing device or system operated by a service provider that receives order information from the server, manages preparation and delivery of ordered food, and provides progress information relating to a delivery state.

The term “actually provided food” refers to a physical food item or a combination of physical food items corresponding to a selected menu item or meal plan, that is prepared and delivered to the user by a provider associated with the external delivery service device.

The term “order information” refers to digital data transmitted from the server to the external delivery service device, the digital data including at least an identification of an actually provided food, a destination information item, and an optional time or quantity parameter, to cause execution of a delivery transaction.

The term “progress information” refers to digital data indicating a state of processing of the order information, including, for example, acceptance, preparation, dispatch, arrival estimation, and completion of delivery, transmitted from the external delivery service device to the server.

The term “evaluation information” refers to digital data expressing a user's assessment of an actually provided food or meal plan, including, for example, numerical ratings, categorical labels, or free-text comments, acquired by the server from the terminal.

The term “nutrition balance conditions” refers to constraints or target values relating to nutritional aspects of a meal plan, including, for example, distributions of nutrients, total energy amount, intake timing, or other dietary parameters that the generative AI model is instructed to satisfy.

The term “adjustment condition” refers to information included in a prompt sentence that specifies how contents of an existing meal plan are to be changed, such as tightening or relaxing nutritional constraints, modifying ingredient preferences, or adapting to a change in the estimated health condition of the user.

The term “feedback loop” refers to a processing structure in which outputs and related information, including evaluation information and progress information, are used to update internal data such as preference information and are then used to generate new prompt sentences for the generative AI model, thereby influencing subsequent meal plan generation in an iterative manner.

According to one embodiment, a server cooperates with one or more terminals and external service devices to implement the invention. The server includes at least one processor, a main memory, and a non-volatile storage device such as a magnetic disk or a solid-state drive. The server is connected to a communication network and is capable of communicating with terminals, external information providing devices, and external delivery service devices using standardized communication protocols such as HTTP over TCP/IP. A terminal operated by a user includes a processor, a memory, a display unit, an input interface such as a touch panel, and one or more sensors or sensor interfaces. The terminal may be, for example, a portable information processing device or a wearable information processing device. The terminal executes an application that collects biometric information and behavioral information of the user and transmits such information to the server. The terminal also receives meal plans, order status information, and feedback requests from the server and displays corresponding information on the display unit.

In one embodiment, the server executes a plurality of functional modules implemented as software components running on an operating system. The software components may be implemented using a programming language such as Python and may utilize numerical computation libraries such as NumPy and scientific data processing libraries such as pandas. The server may also employ machine learning frameworks such as TensorFlow or PyTorch to implement a generative AI model and supporting neural network models used for natural language processing and recommendation tasks.

The server maintains one or more databases, for example, a relational database system, to store user profiles, biometric information, behavioral information, text information, preference information, social situation information, candidate menu information, and order-related information. In one embodiment, the server defines a user profile as a data record including multiple fields such as user identifier, average daily step count, average sleep time, resting heart rate trend indicator, activity level category, and other derived metrics computed from raw sensor data. The server stores these records in a structured table and uses indexed access to efficiently retrieve and update user profiles.

The server collects biometric information and behavioral information by receiving measurement data from terminals and associated sensors. For example, the terminal acquires step counts, heart rate values, and sleep durations from built-in sensors or from a platform health application via an application programming interface. The terminal formats these values in a predefined schema and transmits them to the server at predetermined intervals or upon occurrence of specific events. The server receives the data, verifies integrity, and stores it as time-series records in a storage device. The server then executes an aggregation process using numerical computation libraries to compute user-specific summary features such as a moving average of daily steps over a fixed window, a slope of resting heart rate over several days as a trend metric, and an activity level classification based on threshold rules or a lightweight classifier.

In parallel, the server retrieves text information from external information providing devices, such as social networking platforms, via associated APIs. The server authenticates itself using access tokens and fetches recent postings generated by the user. The server stores raw text in a text table and associates each text entry with a user identifier and timestamp.

The server then executes a natural language processing pipeline implemented using a natural language processing library and a neural network-based model such as a transformer-based encoder. The server tokenizes each text entry, normalizes characters, removes extraneous symbols, and feeds token sequences into a model that has been trained to perform at least two tasks: extraction of food-related entities and extraction of social situation indicators. The neural network model can be a multi-layer transformer network with an attention mechanism, a predefined number of layers, and hidden units. During training, the model receives labeled data containing food-related phrases, positive or negative sentiment labels, and social context labels, and optimizes a cross-entropy loss function using gradient-based methods such as stochastic gradient descent with adaptive learning rate adjustments.

In operation, the server uses the trained model to output, for each text entry, a set of key phrases indicating liked or disliked foods and a set of tags indicating social situations such as eating alone, eating with family, or eating with friends. The server further computes sentiment scores associated with each key phrase by applying a classification head on top of the transformer encoder outputs, yielding numerical values representing the degree of liking or disliking. The server aggregates these values across multiple postings to build preference information. The preference information may be represented as a vector of features where each dimension corresponds to a food category, ingredient type, cooking method, or dietary attribute, and each feature value represents a normalized preference score.

The server also constructs social situation information as a vector of features representing, for example, frequency of group meals, typical meal times with others, and preferred environments (such as at home or at a restaurant). These features are computed by counting occurrence of corresponding labels in the text analysis results and normalizing them over a fixed time window. The server stores both preference information and social situation information as structured records associated with the corresponding user profile. In one embodiment, the server generates a prompt sentence to be input into a generative AI model. The server constructs the prompt sentence by concatenating segments that describe the biometric information, behavioral information, preference information, and social situation information in a standardized natural language template. The server may use rule-based templates and reinforcement-learned templates that have been optimized to produce more stable outputs from the generative AI model. An example of such a prompt sentence is: “The user is a 35-year-old office worker. Recent biometric data from the terminal shows an average of 7,500 steps per day, 6 hours of sleep, and a slightly increasing resting heart rate over the past two weeks. Behavioral information indicates a moderate activity level. Analysis of recent social posts shows that the user wants to eat healthier, avoid fried foods, and enjoys spicy tofu and vegetables, often having dinner with friends on weekends. Based on this information, as a nutrition expert, use a generative AI model to propose three dinner meal plans that are low in calories (under 600 kcal each), nutritionally balanced, and suitable for sharing with friends. For each meal plan, provide a dish name, a short description, main ingredients, and an approximate calorie count.”

The server supplies the prompt sentence to the generative AI model implemented on the same server or on an associated computing resource. The generative AI model may be a transformer-based language model comprising an embedding layer, a plurality of self-attention layers, and an output layer for generating text tokens in sequence. The model is trained on a large corpus including nutrition-related texts, recipes, menu descriptions, and health guidelines. During training, the model optimizes a next-token prediction objective with a cross-entropy loss and uses techniques such as mini-batch gradient descent, weight regularization, and learning rate scheduling. The server may further fine-tune the generative AI model using domain-specific data aligning meal descriptions with nutritional content and user feedback, thereby improving accuracy and responsiveness of generated meal plans to the specific data structures used by the system.

The server configures the generative AI model for inference by specifying generation parameters such as a maximum number of tokens, a temperature parameter controlling randomness, and constraints on output formats. The server then receives from the generative AI model a generated text describing multiple meal plans. The server post-processes the text by identifying dish names, approximate calories, and ingredient lists using a combination of regular expressions and a secondary, lightweight parser model, and converts the information into a structured internal representation. This internal representation may include a field for each dish, containing identifiers, textual description, nutritional estimates, and confidence scores.

The server transmits the resulting meal plan data to the terminal. The terminal receives the data and displays it as a set of selectable items on the display unit. The user views the proposed meal plans and performs a selection input, for example, by tapping on a displayed item. The terminal sends a selection message back to the server. This interaction allows the server to capture the user's choice as a discrete data point that can be used in subsequent learning and optimization.

In order to translate an abstract meal plan generated by the generative AI model into an actual order, the server acquires candidate menu information from an external delivery service device. The external delivery service device provides a catalog of available items, including textual descriptions, prices, and optionally estimated nutritional content. The server retrieves these catalogs via an API, stores them in a candidate menu database, and pre-processes the descriptions by applying term normalization and vectorization, for example using word embeddings or sentence embeddings.

The server then constructs another prompt sentence that includes the abstract meal plan and a summary of candidate items. The server may compress the candidate list into a compact representation by clustering similar items and representing each cluster with representative text snippets. An example of a matching prompt sentence is: “Here is a recommended dinner meal plan: ‘Light Spicy Tofu and Vegetable Hotpot, with plenty of vegetables, low oil, and about 480 kcal.’ Here is a list of available dishes from nearby providers, including dish names and descriptions. As a recommendation engine, choose the one or two dishes from the list that most closely match the recommended meal plan in terms of ingredients (tofu and vegetables), spicy flavor, and low oil content. Return the chosen dish names and identifiers in text form.”

The server supplies this prompt sentence to the generative AI model, which, due to its training on language similarity and structured description alignment, produces an output describing one or more candidate items as the best matches. The server parses this output and associates it with identifiers from the candidate menu database. The server thereby determines the actually provided food items that correspond to the abstract meal plan. The server then composes order information including the selected item identifiers, destination address, and delivery options, and transmits the order information to the external delivery service device. The external delivery service device returns progress information describing the current state of the order, such as accepted, preparing, out for delivery, or delivered. The server stores this progress information in association with the user profile and sends corresponding notifications to the terminal. The terminal displays the progress information to the user in near real-time, using push notification mechanisms. After the food is delivered, the terminal presents a feedback interface to the user. The user inputs evaluation information, such as numerical ratings and textual comments. The terminal transmits this evaluation information to the server. The server stores the evaluation information and uses it to update preference information. In one embodiment, the server processes the textual comments using the same or a similar natural language processing pipeline as applied to social postings, but with labels oriented to post-consumption satisfaction. The server adjusts preference scores for affected food attributes and cooking methods in the user's preference vector. This update process may be implemented as an incremental learning rule, for example, applying a weighted moving average or a small gradient update to a parameterized preference model.

The server then generates a new prompt sentence for the generative AI model that incorporates the updated preference information and possibly includes explicit guidance derived from the feedback. An example of such a feedback-oriented prompt sentence is: “The user previously selected ‘Light Spicy Tofu and Vegetable Hotpot’ but provided the following feedback: ‘Tasty but still a bit oily; I want lighter dishes next time.’ Based on the user's biometric data and updated preferences, propose two dinner meal plans that are less oily and slightly lower in calories, while retaining spicy flavor and tofu as a main ingredient.” By using updated prompt sentences that integrate newly obtained evaluation information, the server forms a feedback loop that allows the system to converge to more accurate and personalized meal plans over time. The use of structured vectors for user profiles, preference information, and social situation information, and the repeated construction of prompt sentences based on these vectors, yields a specific data flow and control structure. This structure reduces repeated parsing of heterogeneous data, because once data are normalized into the internal vector representations and stored in the database, the server can re-use them when forming new prompt sentences without needing to re-scan original text sources or raw sensor streams.

From a technical perspective, the architecture improves computer operation in several ways. First, by centralizing reasoning tasks-meal planning, menu matching, and feedback integration-into a single generative AI model that is controlled via structured prompt sentences, the server reduces the number of distinct, hand-crafted rule modules that would otherwise be required. Traditional systems might implement separate modules for nutritional analysis, preference matching, and item mapping, each with their own parsing and scoring routines. In contrast, the present system organizes these operations as prompt-driven calls to a single generative AI model, thereby lowering the overhead of maintaining multiple algorithms and reducing redundancy in data transformation.

Second, the server's use of normalized vector-based user profiles and candidate menu representations enables efficient similarity computation and preselection of candidates before invoking the generative AI model. The server can filter candidate menu items by comparing their embeddings to the embeddings of target meal plan descriptions using fast linear algebra operations implemented in numerical libraries. This filtering step reduces the length of candidate lists included in matching prompt sentences, thereby reducing latency and bandwidth in communication with the generative AI model and increasing the throughput of the overall system.

Third, by integrating user feedback into the same workflow used for initial recommendation, the server avoids offline retraining cycles that require taking the system out of service or waiting for batch updates. Instead, the server can perform online adaptation by incrementally updating preference vectors and modifying subsequent prompt sentences in real time. This approach reduces the need for complete retraining of the generative AI model, lowers computational costs, and shortens the time between feedback input and system behavioral change.

In one alternative embodiment, the generative AI model may be implemented as a sequence-to-sequence neural network with an encoder-decoder architecture using attention, instead of a pure transformer encoder. The encoder receives a serialized representation of the user profile and preference information, while the decoder generates text tokens representing meal plans or item selections. Training can include multi-task objectives where the model learns to output both free-form descriptions and structured tags indicating nutritional categories. The server may store model parameters in the storage device and load them into memory as needed, enabling runtime switching between models with different sizes or capabilities depending on available computation resources and desired response time.

In another embodiment, the server may employ a hybrid strategy, where a smaller, specialized neural network is used for candidate pre-ranking based on embeddings, and the generative AI model is used only for final explanation and refinement of meal plan text. In this configuration, the server further improves processing speed by delegating simple similarity computations to lightweight models, reserving the generative AI model for tasks that benefit from richer language understanding. This configuration also allows the server to adapt dynamically to network conditions or service loads, by selecting an appropriate balance between computational complexity and output richness.

Because the server systematically controls data acquisition, vectorization, prompt construction, generative inference, and feedback integration using explicit data structures and trained models, the overall system achieves a reduction in communication load, improved consistency of user state across components, and enhanced accuracy in matching abstract recommendations to real-world items. These improvements are not limited to automating a human operator's decision-making, but instead arise from specific computer-technical arrangements, including structured internal representations, learned mapping functions, and prompt-based orchestration of a generative AI model, that collectively enhance the functioning of the server and its associated terminals as information processing machines.

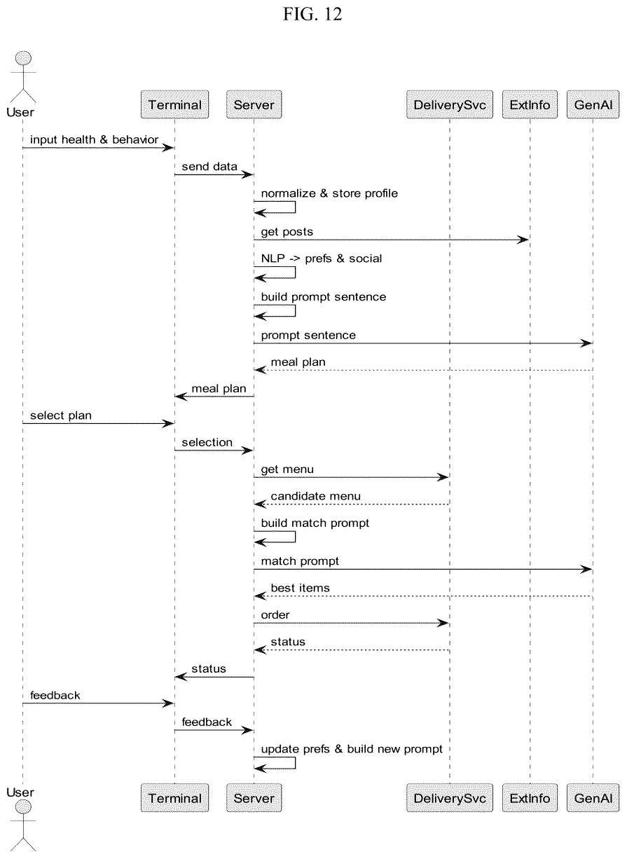

The following describes the processing flow using FIG. 12.

### Step 1

The terminal acquires biometric information and behavioral information of the user.

The terminal uses sensors and platform health APIs to read step counts, heart rate values, sleep durations, and activity logs, and it aggregates these values into a local data structure. The input of this step is raw sensor signals and platform-specific records; the output of this step is a structured data set containing time-stamped biometric and behavioral measurements. The terminal converts the measurements into normalized units, packages them into a predefined schema, and transmits the structured data set to the server via a communication network.

### Step 2

The server receives and standardizes biometric information and behavioral information. The server takes as input the structured data set sent from the terminal and stores the records in a time-series table. The server then performs data cleaning and normalization by using numerical computation libraries to remove invalid values, align timestamps to a common time zone, and convert measurement units. The output of this step is a standardized user profile segment containing averaged values, trend indicators, and categorized activity levels. The server writes this user profile segment into a user profile database associated with a user identifier.

### Step 3

The server acquires text information including posting information of the user from an external information providing device.

The server uses authentication tokens to call an external API and retrieves recent text postings by the user. The input of this step is an API request with the user identifier and access credentials; the output is a collection of text entries with timestamps and metadata. The server stores each text entry in a text information table, linking the entry to the corresponding user identifier for subsequent analysis.

### Step 4

The server analyzes text information to extract preference information and social situation information.

The server reads the stored text entries as input and processes them with a natural language processing pipeline. The server tokenizes each sentence, applies a trained neural network model to identify food-related entities, evaluates sentiment scores for those entities, and detects phrases associated with social contexts such as “with friends” or “family dinner.” The output of this step is a preference information vector and a social situation information vector, each containing numerical or categorical values representing likes, dislikes, and typical social eating patterns. The server stores these vectors as structured records in association with the user profile.

### Step 5

The server constructs a prompt sentence for the generative AI model based on the user profile, preference information, and social situation information.

The server takes as input the standardized user profile segment, the preference information vector, and the social situation information vector. The server formats these data points using a template to generate a natural-language description of the user's health condition, behavioral patterns, food preferences, and social context. The output of this step is a prompt sentence that specifies conditions and constraints for meal planning. The server concatenates profile-derived details with explicit instructions such as calorie limits and number of meal plans, creating a complete prompt sentence for the generative AI model.

### Step 6

The server supplies the prompt sentence to the generative AI model and generates a meal plan.

The server passes the constructed prompt sentence as input to the generative AI model implemented on a computing node. The generative AI model processes the input sequence and outputs a sequence of tokens representing textual descriptions of one or more meal plans. The server receives this generated text as output and parses it into structured elements such as dish names, ingredient descriptions, and approximate calorie counts. The server stores the structured meal plan in a meal plan table and keeps the original generated text for reference.

### Step 7

The server transmits the generated meal plan to the terminal for display and selection.

The server takes as input the structured meal plan created in the previous step and encapsulates it in a response message. The output of this step is a data payload sent to the terminal containing textual descriptions and identifiers of the proposed meal plans. The terminal receives this data, renders each meal plan as a selectable item on the display unit, and allows the user to scroll, view details, and compare options.

### Step 8

The user selects a meal plan via the terminal.

The terminal presents the generated meal plans to the user and waits for user interaction. The input of this step is the set of meal plan items displayed on the screen; the output is a selection message containing an identifier of the chosen meal plan and, optionally, a requested delivery time or additional constraints. The terminal sends this selection message back to the server through the communication network.

### Step 9

The server acquires candidate menu information from an external delivery service device. The server receives the selection message as input and uses the selected meal plan identifier to retrieve the corresponding abstract meal description. The server then requests candidate menu information from an external delivery service device by calling an API that returns a list of available food items near the user. The output of this step is a candidate menu list containing item identifiers, names, and textual descriptions. The server stores this candidate menu list temporarily for further processing.

### Step 10

The server generates a matching prompt sentence for the generative AI model to select actually provided food.

The server takes as input the abstract meal description from the selected meal plan and the candidate menu list obtained from the external delivery service device. The server compresses or summarizes the candidate items if necessary and embeds both the abstract description and the summarized candidate list into a new prompt sentence. The output of this step is a matching prompt sentence that instructs the generative AI model to choose from among the candidate items those that most closely match the abstract meal plan. The server formats the prompt sentence so that it clearly references ingredients, flavor properties, and nutritional characteristics to guide the generative AI model.

### Step 11

The server uses the matching prompt sentence with the generative AI model to specify actually provided food.

The server supplies the matching prompt sentence as input to the generative AI model and requests a selection of one or more items. The generative AI model generates an output text referencing particular candidate items by name or by description. The server parses this output to identify corresponding item identifiers within the candidate menu list. The output of this step is a set of specified actually provided food items that correspond to the selected meal plan. The server records these item identifiers as the basis for order information.

### Step 12

The server transmits order information to the external delivery service device and manages progress information.

The server constructs order information using the specified item identifiers, the user's delivery address, and delivery preferences as input. The server sends this order information to the external delivery service device and receives progress information in response or through subsequent status updates. The output of this step is an updated order record containing order identifiers, current delivery status, and estimated delivery times. The server stores these records and forwards summarized progress information to the terminal so that the terminal can display real-time delivery status to the user.

### Step 13

The user provides evaluation information regarding the actually provided food via the terminal.

The terminal receives confirmation that the delivery has been completed and prompts the user to submit a rating and comments. The input of this step is the user's experience with the delivered food and the displayed feedback interface; the output is evaluation information that includes at least a numerical rating and optional free-text feedback. The terminal sends the evaluation information to the server as structured data linked to the corresponding order and meal plan.

### Step 14

The server updates preference information and generates a new prompt sentence reflecting updated preferences.

The server receives the evaluation information as input and analyzes the numerical rating and free-text comments. The server applies natural language processing to the comment, detects mentions of attributes such as “too oily” or “very satisfying,” and adjusts preference scores for the corresponding food attributes in the preference information vector. The output of this step is an updated preference information vector and, based on it, a new prompt sentence for the generative AI model. The server constructs the new prompt sentence by incorporating the updated preference information into the narrative description of the user's state, thereby preparing the system to generate subsequent meal plans that more accurately reflect the user's evolving preferences.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented diet recommendation techniques generally perform static rule-based matching between user profiles and pre-defined meal templates. Such techniques often treat user health data, lifestyle data, and dietary preferences merely as lookup keys, without dynamically adapting computational models or system behavior based on the user's longitudinal outcomes. As a result, these systems exhibit several technical limitations in terms of data processing, model control, and system-level performance.

First, conventional systems typically construct fixed queries or simple rule sets rather than generating structured prompt sentences for a generative AI model based on rich user-specific data and system constraints. As a consequence, the computing resources of advanced generative models are underutilized, and the system cannot flexibly control the model behavior in accordance with fine-grained health conditions, lifestyle patterns, and nutritional constraints. This leads to suboptimal use of the model's generative capabilities and increases the need for manual tuning of prompts and parameters.

Second, conventional architectures often separate recommendation generation from prediction of future health outcomes. Nutrient information of generated meal plans is not systematically transformed into machine-readable feature vectors, nor is it consistently combined with stored user health and lifestyle information. As a result, the system cannot effectively apply machine learning models to predict future health states, and thus cannot provide feedback signals that could be used to automatically adjust the behavior of the recommendation pipeline. This lack of integrated feature generation and predictive computation limits the system's ability to improve recommendation relevance and to optimize health-related performance metrics over time.

Third, existing systems generally do not implement a robust feedback loop at the system level, in which user satisfaction information and physical condition change information, collected through the user interface, are systematically fed back into both the predictive models and the prompt generation logic. Conventional feedback mechanisms, if they exist, tend to be manual or ad hoc and do not calculate a quantitative deviation between predicted outcomes and actual outcomes. Therefore, the systems fail to automatically reconfigure machine learning models or prompt generation rules, resulting in degraded accuracy, poor personalization, and inefficient utilization of data collected during operation.

Fourth, in many implementations, data flow between input interfaces, storage components, generative AI models, and predictive models is not managed as a coordinated pipeline. Data is often stored in heterogeneous formats and processed with ad hoc scripts, making it difficult to consistently associate user identification, time information, and generated content. This fragmented design prevents the system from maintaining coherent longitudinal records and hampers the computational efficiency and scalability of the overall architecture.

In view of these limitations, there is a need for an improved computer-implemented system that (i) generates structured prompt sentences for a generative AI model directly from stored user attribute, physical, lifestyle, and preference information, together with explicit nutritional constraints; (ii) automatically transforms generated meal plan data into feature data for machine learning-based health outcome prediction; and (iii) implements a closed-loop control scheme that uses user feedback and prediction errors to automatically update prompt generation conditions and model parameters. Such a system would improve the technical functioning of the computer platform itself by optimizing data flow, enhancing model control, and enabling adaptive, data-driven configuration without requiring manual intervention for each individual user adjustment.

The specific processing by the specific processing unit 290 of the data processing device 12 in Example 2 is realized by the following means.

The present invention provides a server comprising a processor and one or more storage devices, the processor being configured to acquire user attribute information, physical information, lifestyle information, and preference information via an input/output interface, convert the acquired information into digital data, and store the digital data in association with identification information and time information in the storage devices; to generate, from the stored digital data, a structured prompt sentence including the extracted user attribute information, physical information, lifestyle information, preference information, and nutritional intake constraints, and to supply the prompt sentence as input to a generative AI model so as to cause the generative AI model to generate meal plan data; to parse nutrient information contained in the meal plan data, combine the parsed nutrient information with the stored user physical information and lifestyle information, and generate feature data; to input the feature data into a machine learning model in order to compute prediction result data representing predicted future health states and lifestyle changes of the user; to integrate the meal plan data and the prediction result data, transform the integrated information into presentation data suitable for display on a user display device, and transmit the presentation data to the user display device; and to acquire, via the user display device, feedback data including user satisfaction information and physical condition change information, store the feedback data in the storage devices, compute a deviation between the prediction result data and actual health and lifestyle transitions derived from the feedback data, and, when the deviation satisfies a predetermined condition, automatically update generation conditions for the prompt sentence and parameters of the machine learning model so that subsequent meal plan data and prediction result data are improved over time. This enables an integrated, computer-implemented pipeline in which user-specific dietary recommendations are generated by a generative AI model under explicit system-controlled constraints, transformed into structured features for predictive computation, and iteratively refined through a closed-loop feedback mechanism that automatically reconfigures both prompt generation logic and predictive model behavior, thereby improving the technical performance, adaptability, and accuracy of the diet recommendation server.

The term “user attribute information” refers to information that characterizes a user's basic profile, including at least one of age, sex, and other demographic or identification-related attributes that are stable over time.

The term “physical information” refers to information indicating a user's physical or physiological state, including at least one of body weight, blood pressure, body mass index, biometric measurements, or other health-related numerical values.

The term “lifestyle information” refers to information indicating patterns of a user's daily activities and habits, including at least one of exercise frequency, sleep duration, work style, or other behavior-related parameters.

The term “preference information” refers to information indicating a user's likes, dislikes, or constraints regarding consumables, including at least one of food preferences, ingredient preferences, taste preferences, or dietary restrictions.

The term “input/output apparatus” refers to any hardware and software combination that enables a user to input information into, and receive information from, a computing system, including at least one of a terminal device, a display device, a touch panel, a keyboard, or a pointing device.

The term “digital data” refers to information encoded in a machine-readable format suitable for processing by a computing system, including structured or unstructured data represented by numerical values, character strings, or data objects.

The term “communication apparatus” refers to a hardware and software combination that enables transmission and reception of digital data between the server and other devices via a communication network, including at least one of a network interface controller, a wireless communication module, or a communication protocol stack.

The term “storage apparatus” refers to a physical or virtual data storage resource accessible by a processor, including at least one of a magnetic storage device, a semiconductor memory device, an optical storage device, or a networked storage service.

The term “identification information” refers to information that uniquely or quasi-uniquely associates digital data with a particular entity or record, including at least one of a user identifier, a device identifier, a session identifier, or a record key.

The term “time information” refers to information representing a time point or time interval associated with an event or data record, including at least one of a timestamp, date, or time range.

The term “prompt sentence” refers to a machine-readable text sequence that conditions the behavior of a generative AI model, including explicit instructions, contextual information, constraints, and desired output formats.

The term “nutritional intake conditions” refers to constraint information related to nutrients and energy in a meal plan, including at least one of total energy intake, nutrient balance, upper or lower limits for individual nutrients, and avoidance or inclusion conditions for specific components.

The term “generative AI model” refers to a machine-implemented probabilistic model that generates output data, such as natural language or structured data, based on input text or other representations, including at least one of a neural network language model, a transformer-based model, or a generative probabilistic model.

The term “meal plan data” refers to digital data representing one or more proposed meals for a user, including at least one of itemized menus, descriptions of foods, serving sizes, and associated nutrient values.

The term “nutrient information” refers to information indicating nutritional properties of one or more consumables, including at least one of energy value, macronutrient amounts, micronutrient amounts, or other diet-related components.

The term “feature data” refers to numerical or categorical representations derived from raw data and suitable for input to a machine learning model, including at least one of vectors, matrices, or feature sets obtained by combining nutrient information with user health and lifestyle data.

The term “machine learning model” refers to a computational model whose parameters are determined or adjusted by a training process using example data, and that is configured to output prediction values or classification results based on input feature data.

The term “prediction result data” refers to digital data output by a machine learning model that represents predicted future states or outcomes, including at least one of future health state indicators, lifestyle change indicators, or risk scores.

The term “display apparatus” refers to a hardware device and associated software for visually presenting information to a user, including at least one of a liquid crystal display, an organic light-emitting display, or a projection display integrated into or coupled to a terminal device. The term “presentation data” refers to digital data that has been formatted or structured for display to a user, including at least one of layout information, textual information, graphical data, or combined multimedia content.

The term “feedback data” refers to digital data representing user responses or outcomes after a recommendation has been provided, including at least one of satisfaction scores, self-reported physical condition changes, or updated health measurements.

The term “generation conditions” refers to parameter values, rules, or constraints that control how a prompt sentence is constructed and how a generative AI model is to behave, including at least one of included fields, constraint thresholds, and instruction phrases.

The term “parameters of the machine learning model” refers to numerical values or configuration settings that define the internal state or behavior of a machine learning model, including at least one of weight values, bias values, hyperparameters, or threshold values.

The term “constraint conditions” refers to explicit restrictions or target ranges applied to model outputs or data generation processes, including at least one of upper and lower bounds for nutrient values, target energy ranges, and inclusion or exclusion rules for specific categories.

The term “degree of deviation” refers to a quantitative measure indicating a difference between prediction result data output by a machine learning model and actual observed outcomes, including at least one of an error value, a distance metric, or a loss value computed over one or more samples.

The term “learning processing of the machine learning model” refers to operations that adjust parameters of a machine learning model using training data, including at least one of initial training, retraining, fine-tuning, or incremental updating.

The term “generation logic of the prompt sentence” refers to a set of procedures or rules executed by a processor to construct a prompt sentence from stored data and constraints, including at least one of template selection, field insertion, condition encoding, and output-format specification.

In one embodiment, a server, a terminal, and a user cooperate to implement the invention. The server comprises at least one processor, a main memory, a non-volatile storage apparatus, and a communication apparatus connected via a bus. The terminal comprises at least a display apparatus, an input/output apparatus such as a touch panel or keyboard, a local processor, a local memory, and a communication module. The user operates the terminal to provide information and to view results generated by the server.

The server executes an operating system, such as a general-purpose server operating system, and an application stack implemented, for example, using a web application framework (such as a generic scripting-language-based framework or a general-purpose application framework) and a data analysis environment (such as a numerical computation library and a data frame library). The server stores user data and generated data in a database management system, such as a relational database. The server communicates with the terminal through a network, such as a packet-switched network, using a transport protocol stack. The server accesses one or more external or internal generative AI models and machine learning models, which may execute on dedicated accelerator hardware, such as a graphics processing unit cluster or a specialized accelerator array.

The server provides a frontend interface, for example through a web-based client or a native mobile application backend, to the terminal. The terminal renders screens that enable the user to enter user attribute information, physical information, lifestyle information, and preference information. The terminal displays, for example, text fields for age and sex, selection controls for exercise frequency and sleep duration, and selection lists for dietary preferences and restrictions. The user operates the terminal by touching the screen, typing text, or selecting options. The terminal converts the user input into structured digital data, for example a key-value map, and transmits the data to the server over an encrypted communication channel.

The server receives the digital data and stores the data in the storage apparatus as one or more records in tables of the database management system. The server associates the records with identification information, such as a user identifier, and time information, such as timestamps indicating when the data was received. The server maintains a data schema in which user attribute information, physical information, lifestyle information, and preference information are stored in normalized or partially normalized tables. The server may maintain indexes on user identifiers and time information to improve query performance, and the server may maintain separate tables for raw input data and for processed feature data.

The server retrieves the stored user attribute information, physical information, lifestyle information, and preference information in order to construct a prompt sentence for a generative AI model. The server uses a prompt generation module implemented in the application layer, which applies template-based construction with rule-based modifications. The server selects a base template string from a template repository based on at least one of user category, target calorie range, and health risk factors inferred from the stored physical information. The server replaces placeholders in the template with specific values of the stored information and appends constraint phrases describing nutritional intake conditions. For example, the server may generate a prompt sentence such as:
“You are a nutrition expert.
Create a one-day meal plan (breakfast, lunch, and dinner) for the following user.
Age: 35 years.
Sex: male.
Exercise: 3 times per week.
Blood pressure: slightly high.
Body weight: 78 kilograms.
Average sleep duration: 6 hours per night.

The goal is to keep total daily calories around 2000 kilocalories, reduce sodium intake, and maintain a balanced ratio of protein, fat, and carbohydrates suitable for mild hypertension.

Avoid deep-fried foods and minimize processed meats.

Provide concrete dish names and short descriptions for each meal.

Ensure that the total sodium intake stays below a recommended upper limit appropriate for slightly high blood pressure.”

The server may generate different prompt sentences for different users by varying the age, sex, exercise pattern, specific health conditions, and dietary constraints. The server may introduce additional constraint phrases for specific targets, such as weight reduction, endurance training support, or glycemic control. The server may also specify an output style requirement in the prompt sentence, such as asking for short descriptions, specific cuisines, or portion size indications.

The server transmits the prompt sentence to a generative AI model. The generative AI model is, in one embodiment, a large-scale neural network language model based on a transformer architecture. The model comprises multiple layers of self-attention mechanisms and feedforward networks, with parameters trained on large-scale text datasets. The model receives the prompt sentence as a sequence of token identifiers, processes the sequence through embedding layers, attention layers, normalization layers, and output projection layers, and generates an output sequence of tokens that the server decodes into a natural language text response. The server can execute the generative AI model locally on GPU hardware or remotely via an external AI service, using an application programming interface. The server sets model parameters, such as decoding temperature, maximum output length, and nucleus sampling parameters, to adjust diversity and determinism of the generated text. The server analyzes the text produced by the generative AI model and extracts meal plan data. For example, the generated text may contain headings “Breakfast”, “Lunch”, and “Dinner” with corresponding dish descriptions. The server uses rule-based parsing, regular expressions, or shallow natural language parsing to identify meal boundaries, dish names, and portion descriptions. The server may maintain a controlled vocabulary or ontology of food items and nutrient mapping rules to map dish descriptions to standardized food codes. The server then uses an internal nutrient database, which associates each standardized food code with nutrient information such as calories, macronutrients, micronutrients, and sodium content, to compute nutritional values of each dish and each meal.

The server aggregates nutrient information for the entire meal plan. The server computes numeric values such as total daily calories, total protein, total fat, total carbohydrates, and total sodium. The server may compute additional indices, such as the ratio of macronutrients, fiber content, and presence of specific micronutrients relevant to known health conditions. The server stores the resulting nutrient information in the database, associating it with the corresponding meal plan data, user identifier, and time information. The server thereby maintains a structured, queryable history of generated meal plans and their nutrient properties.

The server generates feature data for use by a machine learning model. The server constructs one or more feature vectors combining the nutrient information with selected physical information and lifestyle information of the corresponding user. For example, the server may include features such as current systolic and diastolic blood pressure, current body mass index, exercise frequency, average sleep duration, and historical trend features derived from past records. The server normalizes numerical features using precomputed scaling parameters, encodes categorical features using encoding schemes, and arranges the features in a fixed-order vector representation. The server stores these feature vectors as training or inference inputs in a separate feature table in the database.

The server applies a machine learning model to the feature data to obtain prediction result data. In one embodiment, the machine learning model comprises a multi-layer feedforward neural network trained to predict future health states such as estimated blood pressure after a period of adhering to similar meal plans, or expected weight change. The neural network may comprise an input layer that accepts the feature vector, one or more hidden layers with nonlinear activation functions, and an output layer that produces one or more continuous or categorical outputs. The server trains the neural network in an offline stage using historical data comprising feature vectors and observed outcomes. The server defines a loss function, such as mean squared error for regression of continuous health metrics, and uses an optimization algorithm, such as stochastic gradient descent or an adaptive variant, to update the network weights. The server may augment the training data by applying data augmentation techniques, such as noise injection or synthetic variation of nutrient distributions within clinically reasonable ranges, to improve model robustness. The server periodically retrains or fine-tunes the machine learning model using newly accumulated data, including feedback data from users that includes measured health indicators and self-reported physical condition changes. The server derives labels for training by associating predicted outcomes with subsequent actual measurements of the user. The server computes a degree of deviation between the predictions and actual outcomes using the loss function, and if the deviation exceeds a threshold or if performance metrics degrade, the server triggers a retraining process that updates the model parameters and potentially adjusts model hyperparameters.

The server converts prediction result data into information suitable for presentation. For example, the server may generate textual explanations that relate predicted changes, such as “estimated reduction of systolic blood pressure by approximately 3 mmHg over 4 weeks,” to the nutrient properties of the meal plan, such as reduced sodium and adjusted macronutrient balance. The server may also generate visual indicators, such as risk level color codes or trend arrows, to support user understanding. The server constructs a data structure containing the meal plan descriptions, the nutrient information, and the prediction result data, and transmits this data structure to the terminal.

The terminal receives the data structure from the server and renders it on the display apparatus. The terminal displays, for example, separate sections for breakfast, lunch, and dinner, each showing dish names and short descriptions. The terminal displays numeric nutrient information in tabular or graphical form, and displays the predicted health impact in concise textual form. The user reads the presented information and decides whether to adopt the suggested meal plan. The user may select options to accept, modify, or reject individual meals, and may provide additional feedback such as taste satisfaction and perceived physical effects.

The terminal acquires feedback input from the user and transmits feedback data to the server. The server stores the feedback data in association with the corresponding meal plan, the prediction result data, and the subsequent health measurements, if available. The server uses this feedback data for two distinct but related adaptations. First, the server incorporates the feedback data as additional training data for the machine learning model, thereby refining prediction accuracy and reducing deviation. Second, the server uses the feedback data to update the generation conditions of the prompt sentence. For example, if the user consistently reports low satisfaction with certain ingredient types or meal formats that nonetheless satisfy nutrient constraints, the server modifies the prompt generation logic to de-emphasize those ingredient types or to request alternative preparation methods.

The server implements the prompt generation logic as a set of structured rules and templates, rather than a simple static string. The server may maintain, for example, a library of constraint rules mapping ranges of health indicators to specific textual instructions. If a user exhibits consistently high sodium intake across plans, the server adds a rule that appends explicit phrases such as “significantly limit sodium-rich condiments and processed foods” to the prompt sentence. If the prediction result data indicates insufficient progress toward a weight goal, the server may adjust the target calorie range and insert corresponding text into the prompt sentence. This rule-based adaptation operates at the system level and is not merely a manual selection process. The server thereby achieves automatic, data-driven modification of how the generative AI model is conditioned.

The described architecture improves computer technology in several ways. The server coordinates data flow among multiple specialized modules: input acquisition, storage, prompt generation, generative AI model interaction, nutrient analysis, feature construction, machine learning prediction, and feedback-based adaptation. By storing user data, meal plan data, nutrient information, feature data, and feedback data in structured, indexed tables and by associating each with identification and time information, the server enables efficient retrieval and temporal analysis at scale, reducing computational overhead for repeated recommendation cycles.

The server uses numerical feature vectors that integrate generative AI outputs with structured health and lifestyle data, which allows the machine learning model to capture complex nonlinear relationships that are not apparent from rule-based systems. Because the server separates the generative AI model, which synthesizes meal descriptions, from the predictive model, which evaluates long-term health impact, and because it ties them together through defined data structures and feature transformations, the system can achieve more accurate and computationally efficient predictions than systems that attempt to directly encode all logic in a single monolithic model.

The closed-loop control implemented by the server improves prediction accuracy and recommendation relevance over time while reducing the need for manual intervention. The server calculates a degree of deviation between prediction result data and actual outcomes and automatically triggers retraining or fine-tuning of the machine learning model and adjustment of prompt generation rules. This process not only reduces prediction errors but also maintains stable computation time by adjusting model complexity and hyperparameters according to accumulated data volume and performance metrics.

The architecture also improves communication efficiency. By generating structured prompt sentences that include only the necessary user attributes, health indicators, and constraints, rather than transmitting entire raw medical records or large unstructured datasets, the server reduces the amount of data that must be transmitted to the generative AI model service. This minimizes communication load and shortens end-to-end response time, providing a technical improvement in network usage and latency.

The described system is not limited to a single embodiment. In another embodiment, the server uses a different type of machine learning model, such as a gradient-boosted decision tree ensemble, for predictive tasks. In yet another embodiment, the server executes the generative AI model on-premises using locally installed transformer models, with weight matrices stored on a dedicated accelerator card, and the server manages model loading, caching, and batching of requests to improve throughput. In still another embodiment, the server maintains multiple prompt templates for different device capabilities, such as generating shorter textual explanations and simplified meal descriptions for terminals with limited display resolution or lower processing capacity.

The system can also be extended to control additional devices. For example, the server may transmit summarized meal information and nutrient targets to a kitchen appliance controller, such as a smart oven or cooker, to preconfigure cooking modes or timing based on the recommended meal plan. In such a case, the server translates generative AI outputs into structured control parameters for the appliance, and the system uses feedback from appliance sensors, such as temperature and cooking completion times, as part of the data used for future prediction and adaptation. This demonstrates that the generative AI-based computation is not merely an abstract mental process but is used to drive specific technical operations of physical devices.

By designing the data structures, model architectures, training methods, and rule-based prompt generation logic as described, the server improves the speed, accuracy, and robustness of diet-related computation compared to conventional systems. The integration of generative AI outputs, structured nutrient databases, feature engineering, and adaptive machine learning leads to reduced computational redundancy, more accurate forecasts, improved data management, and reduced communication load, thereby constituting an improvement to the functioning of the computer-based diet recommendation server itself.

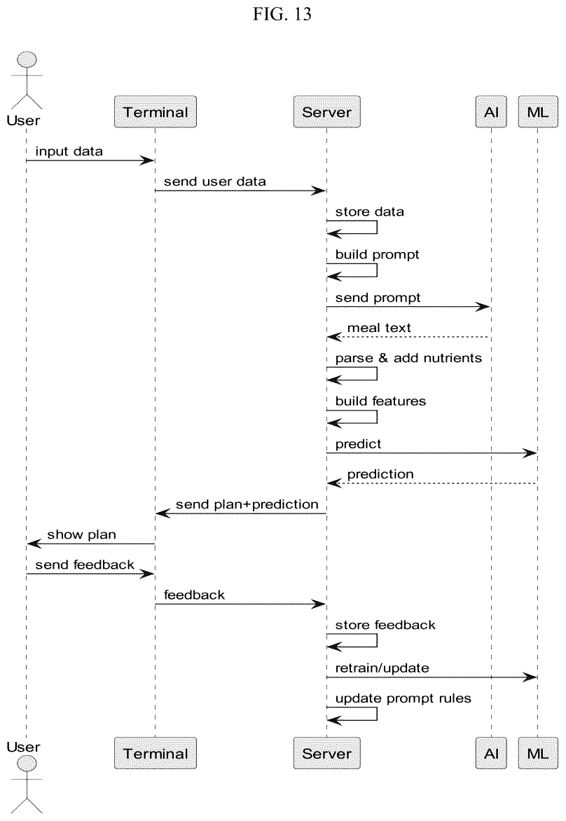

The following describes the processing flow using FIG. 13.

### Step 1

The terminal displays input screens for user attribute information, physical information, lifestyle information, and preference information. The terminal uses its display apparatus and input/output apparatus to show text fields, selection lists, and sliders.

Input: A screen template and existing stored values (if any) received from the server.

Processing: The terminal renders input components and associates each component with an internal field identifier.

Output: A visible user interface that enables the user to enter or modify data.

### Step 2

The user operates the terminal to input values such as age, sex, weight, blood pressure, exercise frequency, sleep duration, and dietary preferences or restrictions.

Input: The visible user interface of Step 1.

Processing: The user touches the screen or uses keys to fill fields and confirms by pressing a submit control.

Output: Raw input events and filled field values stored temporarily in the terminal memory.

### Step 3

The terminal validates the input values and converts them into a structured digital representation.

Input: Raw field values edited by the user in Step 2.

Processing: The terminal checks data types (for example, integer for age, numeric ranges for blood pressure), applies basic consistency rules (for example, non-empty mandatory fields), and encodes the values into a key-value structure such as a map or record.

Output: Structured digital data representing user attribute information, physical information, lifestyle information, and preference information.

### Step 4

The terminal transmits the structured digital data to the server via a communication network.

Input: Structured digital data from Step 3.

Processing: The terminal constructs a message payload, attaches a user identifier or session identifier, and sends the payload using a network protocol over a secure channel.

Output: A network message containing the user data delivered to the server.

### Step 5

The server receives the network message and stores the digital data in a storage apparatus. Input: The network message from Step 4 containing user data and identification information.

Processing: The server parses the message, extracts the user identifier, timestamps the reception, and inserts the data into one or more database tables, associating the data with identification and time information.

Output: Database records containing normalized user attribute information, physical information, lifestyle information, and preference information linked to the user.

### Step 6

The server retrieves the latest stored information for a target user and constructs a prompt sentence for a generative AI model.

Input: User identifier and database records created in Step 5.

Processing: The server executes queries to obtain current attribute, physical, lifestyle, and preference data, selects a prompt template based on health conditions and target nutritional goals, replaces placeholders with actual values, and appends constraint phrases for nutritional intake.

Output: A complete prompt sentence in natural language that encodes user context and nutritional constraints.

### Step 7

The server transmits the prompt sentence to a generative AI model and obtains generated meal plan text.

Input: The prompt sentence from Step 6.

Processing: The server packages the prompt sentence into a model request, sets model parameters such as temperature and maximum length, and sends the request to a model execution environment. The generative AI model processes the tokenized prompt and returns an output token sequence, which the server decodes into text.

Output: Generated text describing a meal plan, typically including breakfast, lunch, dinner, and optional explanations.

### Step 8

The server parses the generated meal plan text and converts it into structured meal plan data. Input: The generated text from Step 7.

Processing: The server applies parsing rules and pattern matching to identify meal sections, dish names, and preparation notes, normalizes dish descriptions, and creates a structured representation containing at least meal type and dish description fields.

Output: Structured meal plan data representing each meal and its dishes.

### Step 9

The server maps each dish in the structured meal plan data to nutrient information using a nutrient database.

Input: Structured meal plan data from Step 8 and entries in a nutrient database.

Processing: The server matches dish descriptions to standardized food entries, retrieves nutrient profiles for matched entries, and associates nutrient values such as calories, macronutrients, and sodium with each dish and meal.

Output: Meal plan data enriched with nutrient information at the dish and meal levels.

### Step 10

The server aggregates nutrient information to compute total daily nutrient values and derived indicators.

Input: Nutrient-enriched meal plan data from Step 9.

Processing: The server sums nutrient values across dishes and meals, calculates totals and averages, and computes indicators such as macronutrient ratios, sodium totals, and comparison against recommended ranges.

Output: Aggregated nutrient data and indicator values describing the nutritional profile of the generated meal plan.

### Step 11

The server constructs feature data by combining aggregated nutrient data with user health and lifestyle data.

Input: Aggregated nutrient data from Step 10 and stored physical and lifestyle information from the database.

Processing: The server selects relevant variables, normalizes numeric values, encodes categorical attributes, and arranges them into one or more feature vectors suitable for a machine learning model.

Output: Feature data representing the joint state of the generated meal plan and the user's current health and lifestyle conditions.

### Step 12

The server applies a machine learning model to the feature data to predict future health states and lifestyle changes.

Input: Feature data from Step 11 and model parameters from stored model definitions.

Processing: The server loads or references the machine learning model, feeds the feature vectors through the model's layers, and computes predicted outputs such as expected blood pressure, weight change, or risk indicators.

Output: Prediction result data representing estimated future health states and lifestyle changes associated with the meal plan.

### Step 13

The server integrates meal plan data and prediction result data into presentation data for the user.

Input: Structured meal plan data from Step 8 or Step 9, aggregated nutrient data from Step 10, and prediction result data from Step 12.

Processing: The server formats textual descriptions of meals, summarizes key nutrient values, generates explanatory sentences linking nutrient patterns to predicted outcomes, and constructs a display-ready data structure.

Output: Presentation data containing meals, nutrients, and predicted impacts formatted for rendering on the terminal display.

### Step 14

The server transmits the presentation data to the terminal.

Input: Presentation data from Step 13.

Processing: The server serializes the presentation data into a transmission format, attaches addressing and session information, and sends the data to the terminal over the communication network.

Output: A network response containing presentation data received by the terminal.

### Step 15

The terminal renders the presentation data on the display apparatus for the user.

Input: Presentation data from Step 14.

Processing: The terminal parses the data, maps meal descriptions to visual components, displays nutrient values in tables or charts, and renders prediction summaries as text or icons.

Output: A user interface presenting the generated meal plan, detailed nutrient information, and predicted health and lifestyle impact.

### Step 16

The user reviews the presented information and optionally modifies or confirms the meal plan.

Input: The rendered user interface from Step 15.

Processing: The user inspects meals and predictions, decides whether to follow the plan, and may select options such as confirm, adjust, or request alternatives.

Output: User interaction events and possible modification commands captured by the terminal.

### Step 17

The terminal collects the user's satisfaction information and physical condition change information as feedback data after the meal plan has been used.

Input: User interaction screens for feedback and user-entered feedback values.

Processing: The terminal displays feedback forms, receives ratings, comments, and updated health measurements, and packs them into a structured feedback record.

Output: Structured feedback data representing satisfaction levels, self-reported physical changes, and updated measurements.

### Step 18

The terminal transmits the feedback data to the server.

Input: Structured feedback data from Step 17.

Processing: The terminal encodes the feedback into a message, associates it with the relevant meal plan identifier, and sends it to the server via the communication network.

Output: A feedback message containing user feedback data delivered to the server.

### Step 19

The server stores the feedback data and associates it with prior meal plans and prediction results.

Input: Feedback message from Step 18 and stored records of meal plans and prediction result data.

Processing: The server parses the feedback message, links the feedback to specific meal plan identifiers and timestamps, and inserts the feedback into feedback tables, maintaining references to prediction records and user records.

Output: Persisted feedback records that can be used for model evaluation and retraining.

### Step 20

The server computes a degree of deviation between prior prediction result data and actual health and lifestyle changes inferred from the feedback data.

Input: Prediction result data from Step 12 and feedback records from Step 19.

Processing: The server aligns prediction time points with observed outcomes, calculates errors or distance measures for each predicted metric, and derives overall deviation metrics or loss values.

Output: Deviation data indicating discrepancies between model predictions and actual outcomes.

### Step 21

The server updates the machine learning model and the prompt generation conditions based on the deviation data and feedback data.

Input: Deviation data from Step 20, feedback records from Step 19, and current model and prompt configuration.

Processing: The server determines whether deviation exceeds thresholds, triggers retraining or fine-tuning of the machine learning model if necessary, updates model parameters to reduce future error, and modifies rules or templates used in prompt sentence generation to better reflect user preferences and observed responses.

Output: Updated machine learning model parameters and updated prompt generation rules that will be applied in subsequent executions.

### Step 22

The server uses the updated machine learning model and updated prompt generation conditions in subsequent iterations to improve the quality and accuracy of generated meal plans and predictions.

Input: Newly entered user data and updated models and rules from Step 21.

Processing: The server repeats Steps 6 through 13 with refined prompt sentences, more accurate features, and improved predictive behavior, thereby adjusting outputs in accordance with accumulated evidence.

Output: Refined presentation data in later cycles, exhibiting improved alignment with user preferences and health outcomes.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional meal recommendation systems generally rely on static rule sets or shallow preference filters that operate on limited user attributes such as basic demographic information and coarse dietary preferences. These systems typically treat input data as simple content parameters and do not use such data to improve the operation of the underlying computing components themselves. As a result, existing systems suffer from several technical limitations.

First, conventional systems do not integrate heterogeneous, time-varying user signals, including physical information, lifestyle information, preference information, and emotional information, into a unified, machine-processable representation that can be efficiently exploited by a generative artificial intelligence model. Separate handling of these data types leads to fragmented storage schemes, redundant processing, and inefficient use of memory and processor resources, as well as difficulty in maintaining temporal consistency of user state in the database.

Second, in many existing approaches, interaction with a generative artificial intelligence model is performed using manually written, ad-hoc prompts that are not systematically derived from structured user data. Such prompts do not encode explicit constraints relating to nutrients, energy intake, or intake restrictions, and therefore cannot reliably guide the generative model to produce outputs that satisfy health-related constraints. This results in a technical mismatch between the structured data maintained by the system and the unstructured input expected by the generative model, causing additional conversion overhead and limiting the accuracy and controllability of generated content.

Third, conventional architectures generally lack an integrated feedback mechanism in which user reactions (acceptance or rejection of proposed meals) are captured in a structured form and used as positive and negative training signals to update the behavior of the generative model or the prompt generation process. As a consequence, the computing system does not adapt its internal models over time in a technically meaningful way; the system state (including model parameters and prompt-generation logic) remains largely static even as new data accumulates, leading to degraded relevance and inefficient use of stored reaction data.

Fourth, typical systems provide only qualitative or heuristic indications of potential health effects of recommended meals. They do not tightly couple generative outputs with numerical prediction pipelines that use nutritional attributes and time-series health and lifestyle data to compute quantitative forecasts of the effect of proposed meals on a user's health state. This lack of coupling prevents the processor from leveraging predictive computation to refine subsequent prompts and model behavior, and therefore fails to exploit available computational resources to improve the technical performance of the recommendation pipeline.

Accordingly, there is a need for a computing system that (i) acquires and normalizes diverse user data, (ii) automatically generates structured prompt sentences that embed constraint conditions derived from such data, (iii) invokes a generative artificial intelligence model in a controlled manner to generate meal proposal information, (iv) performs numerical prediction of health impact based on the generated proposals, and (v) implements a closed-loop feedback mechanism that updates operational characteristics of at least one of the generative model and the prompt generation process based on user reactions and time-series data. Such a system should improve the way the processor stores, transforms, and uses user-specific data to drive generative inference, thereby enhancing the technical functioning of the computer system itself rather than merely automating a human mental process.

The specific processing by the specific processing unit 290 of the data processing device 12 in Application Example 2 is realized by the following means.

The present invention provides a server comprising a processor and a storage device, the processor being configured to acquire, via a user information input/output apparatus, user information including physical information, lifestyle information, preference information, and emotional information; convert the acquired user information into a standardized digital data format and transmit the standardized digital data via a communication network; receive, via the communication network, the transmitted digital data and store the digital data in an information storage region of the storage device such that the stored digital data is managed in association with each user in chronological order; calculate, based on the stored physical information, lifestyle information, preference information, and emotional information, feature values representing at least a health state, a lifestyle tendency, a preference tendency, and an emotional state of the user; automatically generate, using the feature values, a prompt sentence in a natural language; input the prompt sentence to a generative artificial intelligence model and cause the generative artificial intelligence model to generate, in the natural language, meal proposal information that takes into account the health state, the lifestyle tendency, the preference tendency, and the emotional state of the user; extract nutritional information and intake amount information from the meal proposal information output from the generative artificial intelligence model, combine the nutritional information and the intake amount information with the physical information and the lifestyle information, and numerically predict an influence of the meal proposal information on the health state of the user; present the meal proposal information and a prediction result of the influence to a display device corresponding to the user information input/output apparatus, and acquire, from the user, reaction information indicating acceptance or rejection with respect to the meal proposal information; and register the reaction information in the storage device as positive example data and negative example data and update an operational characteristic of at least one of the generative artificial intelligence model and a prompt sentence generation process by using the positive example data and the negative example data so as to adapt contents of generation of subsequent meal proposal information to the preference tendency and the emotional state of the user. This enables the server to technically improve operation of the computing system by (i) unifying heterogeneous user data into structured feature representations used to construct machine-optimized prompt sentences, (ii) constraining and guiding the generative artificial intelligence model through these dynamically generated prompt sentences so as to produce outputs satisfying health-related and user-specific constraints, (iii) numerically linking generative outputs with predictive computation of health impact to refine downstream processing, and (iv) implementing a closed-loop learning mechanism that continuously updates internal model behavior and prompt generation logic based on structured positive and negative feedback, thereby enhancing system-level efficiency, adaptability, and accuracy in generating user-specific meal proposal information.

The term “user information” refers to information associated with an individual user, including, but not limited to, physical information, lifestyle information, preference information, and emotional information that is acquired, stored, and processed by the system. The term “physical information” refers to data indicative of a physical state of a user, including, but not limited to, body weight, body height, age, biological sex, biometric measurements, medical conditions, and other physiological parameters.

The term “lifestyle information” refers to data indicative of habitual behaviors or daily activities of a user, including, but not limited to, exercise frequency, step counts, sleep patterns, work schedules, and other activity-related records.

The term “preference information” refers to data indicative of tastes or choices of a user, including, but not limited to, favored or disfavored food types, cuisine categories, dietary styles, budget ranges, and context-dependent selection tendencies.

The term “emotional information” refers to data indicative of an affective or psychological state of a user, including, but not limited to, self-reported mood descriptions, text expressions, voice characteristics, facial expressions, and derived emotion labels.

The term “user information input/output apparatus” refers to an apparatus that provides an interface for a user to input and receive information, including, but not limited to, a computing terminal such as a smartphone, a tablet, a personal computer, or a wearable device, together with associated input and display components.

The term “standardized digital data format” refers to a data representation in which heterogeneous user information is normalized into a consistent, machine-processable structure, such as a structured record or a serialized object having predefined keys and value types.

The term “communication network” refers to a data transmission infrastructure, including, but not limited to, wired networks, wireless networks, and the internet, through which digital data is transmitted between the user information input/output apparatus and the server.

The term “storage device” refers to a hardware or logical component that stores digital data, including, but not limited to, semiconductor memory, magnetic storage, optical storage, and database storage systems.

The term “information storage region” refers to a logical region or data structure within the storage device that is allocated to store user information, intermediate processing results, model parameters, and related data.

The term “feature values” refers to numerical or categorical values computed from stored user information, the feature values representing abstracted characteristics of at least a health state, a lifestyle tendency, a preference tendency, and an emotional state of a user.

The term “health state” refers to a condition of physical well-being of a user at a given time, characterized by one or more feature values derived from physical information and lifestyle information.

The term “lifestyle tendency” refers to a pattern or trend in habitual behaviors of a user over time, characterized by one or more feature values derived from lifestyle information.

The term “preference tendency” refers to a pattern or trend in choices or selections made by a user over time, characterized by one or more feature values derived from preference information and reaction information.

The term “emotional state” refers to a current affective condition of a user, characterized by one or more feature values derived from emotional information.

The term “prompt sentence” refers to a text in a natural language that encodes, in a structured manner, feature values and constraint conditions, and that is provided as input to a generative artificial intelligence model to guide generation of output information.

The term “generative artificial intelligence model” refers to a computational model that receives a prompt sentence as input and automatically generates output data, including natural-language text, by probabilistic or learned inference based on previously trained parameters.

The term “meal proposal information” refers to information generated by the generative artificial intelligence model in response to a prompt sentence, the information including proposed dishes, meal plans, or menus, optionally accompanied by explanatory text.

The term “nutritional information” refers to data describing nutritional characteristics of a meal, including, but not limited to, quantities of macronutrients, micronutrients, dietary fiber, and other nutrition-related values.

The term “intake amount information” refers to data describing quantities associated with consumption of a meal, including, but not limited to, portion sizes, total energy intake, and per-meal or per-day intake amounts.

The term “influence of the meal proposal information on the health state” refers to a numerical prediction or estimation of how consumption of a proposed meal or meal plan is expected to change at least one aspect of a user's health state over a given time period.

The term “display device” refers to a device that visually presents information to a user, including, but not limited to, a display panel of a smartphone, a monitor of a personal computer, or a screen of a wearable device.

The term “reaction information” refers to data indicating a user's response to meal proposal information, including, but not limited to, explicit acceptance, explicit rejection, selection of certain proposals, and optional user comments.

The term “positive example data” refers to reaction information and associated context that indicate that a user accepted or favored particular meal proposal information, and that is used as training data to reinforce similar future behavior of a model or process.

The term “negative example data” refers to reaction information and associated context that indicate that a user rejected or disfavored particular meal proposal information, and that is used as training data to discourage similar future behavior of a model or process.

The term “operational characteristic” refers to a parameter, rule set, or behavior pattern of at least one of the generative artificial intelligence model and a prompt sentence generation process, where modification of the operational characteristic changes how outputs are generated.

The term “prompt sentence generation process” refers to a computational process that constructs a prompt sentence by transforming feature values and constraint conditions into a natural-language text suitable for input to the generative artificial intelligence model.

The term “constraint conditions” refers to conditions embedded into a prompt sentence that restrict or guide possible outputs of the generative artificial intelligence model, including, but not limited to, limits on nutrient amounts, energy amounts, and intake restriction conditions. The term “meal plan information” refers to information defining one or more meals over a predetermined period, including meal types, timing, constituent dishes, and associated nutritional and intake attributes.

The term “intake history” refers to stored information indicating what meals, meal components, or meal plans have been consumed or accepted by a user over time.

The term “time-series information” refers to user information stored together with timestamps or temporal indices, such that changes in physical information, lifestyle information, or emotional information over time can be analyzed.

The term “feedback loop” refers to a processing structure in which outputs and associated user reactions are stored and subsequently used as inputs to update at least one of a generative artificial intelligence model and a prompt sentence generation process, thereby influencing future outputs.

In one embodiment, a server cooperates with one or more terminals used by a user to implement a system that generates personalized meal proposal information using a generative AI model guided by a structured prompt sentence. The server includes at least one processor, a storage device, a communication interface, and memory storing executable instructions.

The terminal includes a computing device such as a smartphone, tablet, wearable device, or personal computer, equipped with an input unit (touchscreen, microphone, camera, keyboard) and a display unit.

Server executes a program implemented, for example, in a high-level programming language such as Python running on an operating system. Server uses software components including, but not limited to, a web framework (such as a generic web framework), a database management system (such as a relational database engine), numerical computation libraries (such as a matrix computation library and a data frame processing library), and machine-learning frameworks (such as a deep learning framework with a neural-network API). Server also uses an emotion analysis library (for example, a natural language processing toolkit) and a generative AI model (for example, a large language model accessible through an API or locally deployed).

Terminal acquires user information including physical information, lifestyle information, preference information, and emotional information. Terminal presents input screens on the display unit for the user to enter age, height, weight, gender, known medical constraints, dietary restrictions, favorite cuisines, disliked ingredients, typical activity level, and current mood. Terminal acquires this information via touch input or voice input, normalizes units (for example, kilograms versus pounds), and records timestamps. Terminal also acquires sensor data via short-range communication from devices such as wearable sensors, including heart rate, step counts, and sleep durations. Terminal aggregates these heterogeneous inputs into structured records stored in memory.

Terminal converts user information into a standardized digital data format, such as a hierarchical key-value representation, and transmits this data to the server via a communication network using a secure protocol. By standardizing and compressing the data into a well-defined structure before transmission, the terminal reduces communication overhead and ensures that the server receives only the subset of information needed for subsequent processing, thereby reducing network load.

Server receives the digital data through the communication interface and parses the standardized payload into internal data structures. Server stores the data in a storage device managed by a database system. Server maintains multiple logical tables or collections, including a user profile region, a health record region, a lifestyle record region, a preference record region, an emotion record region, a feedback record region, and a model configuration region. Server stores each record together with a timestamp and associates it with a user identifier. By organizing data into normalized relational tables or equivalent structured stores, server enables efficient indexing and retrieval by user and by time, which reduces query latency and I/O load when constructing feature values.

Server calculates feature values representing the health state, lifestyle tendency, preference tendency, and emotional state of the user. Server loads raw records into in-memory data frames and executes numerical transformations. For physical information, server computes body mass index from weight and height, applies moving averages to weight, and computes deltas over defined time windows to detect gain or loss trends. For lifestyle information, server computes summary features such as average daily step count, weekly step variance, mean and variance of sleep duration, and detects anomalies such as sudden drops in activity.

For preference information, server maintains counts and normalized frequencies of accepted and rejected cuisines, ingredients, and preparation styles, and computes preference scores such as “Japanese cuisine score” or “spicy-food penalty.” For emotional information, server applies a text-processing pipeline using a tokenizer and vectorizer to convert user-submitted text and social posts into token sequences. Server then supplies these token sequences to an emotion classifier network, such as a multi-layer neural network or a transformer-based classifier implemented in a deep learning framework.

In one embodiment, server implements the emotion classifier as a neural network with an embedding layer, one or more bidirectional recurrent layers or transformer layers, and a dense output layer with a softmax activation. Server initializes the network with pre-trained weights or trains it on labeled emotion data. Server uses a loss function such as cross-entropy and an optimization method such as stochastic gradient descent with adaptive moment estimation to train the network. During inference, server feeds token embeddings to the network, obtains class probabilities for multiple emotion categories (for example, “happy,” “sad,” “stressed,” “relaxed”), and selects the highest-probability label as the current emotional state. Server stores both the label and the probability distribution as feature values. By representing user state as a compact vector of features, server improves computation speed and reduces memory usage when generating prompts and performing predictions. Server generates a prompt sentence in natural language using the feature values as inputs. Server does not rely on manually written, static prompts; instead, server uses a prompt generation module that composes text segments based on the computed features and constraint conditions. Server holds template strings with placeholders for age, height, weight, activity metrics, dietary restrictions, preference scores, and emotion labels. Server dynamically fills these placeholders with actual feature values and also appends constraint clauses specifying nutrient targets, maximum caloric intake, and forbidden ingredients. As a result, the prompt sentence precisely encodes both user context and computational constraints into a form that the generative AI model can interpret, which improves controllability and reduces the need for post-hoc filtering.

In one example, server generates a prompt sentence such as:
“User profile: age 40, height 165 cm, weight 72 kg, goal is gradual weight loss.
Health and lifestyle: low activity, often feels tired, sleeps about 5 hours per night. No diabetes, mild hypertension.
Dietary preferences: likes Japanese food and pasta, dislikes spicy dishes, no allergies.
Emotion: currently feels ‘depressed’ with high intensity.
Context: dinner alone at home, low cooking effort preferred.
Constraints: total dinner energy should be around 600 kcal, with at least 20 g of protein and low saturated fat.
Task: Using the generative AI model, propose three dinner options that are low in calories but emotionally comforting. For each option, describe approximate calories, main nutrients, and how it may affect weight and mood.”

In another example, server generates a prompt sentence such as:
“User says: ‘I recently gained weight, so I want lower-calorie meals.’
User health data: weight increased from 70 kg to 74 kg in the last two months, height 170 cm, mild hypertension.
Preferences: likes Japanese food, dislikes spicy food.
Constraints: daily energy intake target 1800-2000 kcal, balanced macronutrients, low salt.
Task: Propose three low-calorie, nutritionally balanced meal options for today's dinner, and briefly explain how each option helps weight control.”

Server supplies the prompt sentence as input to a generative AI model. In one embodiment, server accesses a large language model provided over a network via an application programming interface. In another embodiment, server hosts a generative model locally using a deep learning framework. The generative model can be a transformer-based network with multiple attention layers, positional encodings, and a large vocabulary, trained on general text and optionally fine-tuned on nutrition and recipe corpora.

Server configures generation parameters such as maximum output length, sampling temperature, and nucleus sampling threshold. Server sends the prompt sentence and receives natural-language output describing one or more proposed meals. The generative model constructs each token based on learned conditional distributions using attention mechanisms and neural activations, which differ fundamentally from conventional rule-based systems. Because the prompt embeds structured constraints, the generative model tends to propose meals satisfying calorie, nutritional, and restriction conditions without separate manual checks, thereby reducing computational overhead.

Server then parses the meal proposal information. Server applies a natural language parser to identify dish names, ingredients, portion sizes, and approximate calories. If the generative output already includes calorie estimates, server uses them; otherwise, server matches ingredients and quantities against a nutrition database. Server aggregates nutrient vectors for each dish, computing macronutrients, micronutrients, total energy, and other attributes.

Server combines these derived nutritional vectors with the user's current feature values and supplies them to a predictive model.

In one embodiment, server implements the predictive model as a neural network mapping meal nutrition vectors and user state vectors to quantitative changes in health metrics, such as short-term weight change, approximate blood pressure effect, or fatigue level. The predictive model can be an LSTM network for time-series data, a feed-forward network, or a hybrid architecture. Server trains the predictive model by minimizing a loss function, for example mean squared error between predicted and actual weight changes, using historical intake data and health records stored in the storage device. Server updates weights using gradient-based optimization and can apply regularization to avoid overfitting. This configuration allows server to perform approximate, but consistent, numeric forecasting beyond what a human nutritionist would practically compute in real time.

Server calculates a predicted influence value, for example “estimated weekly weight change −0.3 kg if such dinners are eaten daily” or “likely to reduce subjective fatigue score by 10% over two weeks.” Server appends these predictions to the meal proposal information as structured annotations. The combination of generative text and numeric prediction creates a unified representation that the terminal can display concisely while allowing the server to perform further optimization.

Terminal receives the enriched meal proposal information from the server. Terminal renders a user interface showing, for each proposal, the dish name, summarized nutritional characteristics, approximate calories, predicted influence on weight and other health indicators, and comments on emotional suitability. Terminal may provide visual indicators, such as colored icons or bars, to represent predicted health impact. The user reviews these proposals and selects one or more responses: acceptance for meals the user intends to follow, rejection for unsuitable meals, or ordering through a linked delivery channel where available. Terminal sends feedback to the server, indicating which proposals were accepted or rejected and optionally providing textual comments. Terminal may also record whether an order was placed through an integrated supply service. By collecting this feedback consistently, terminal and server establish a positive/negative sample dataset that reflects user-specific acceptance patterns.

Server receives the feedback and records each event in the feedback record region along with metadata such as the prompt sentence used, the generated meal content, and the user state at the time. Server flags accepted proposals as positive example data and rejected proposals as negative example data. Server uses these labeled examples to adjust internal models and prompt generation logic.

In one embodiment, server trains a ranking model that predicts acceptance probability for candidate meals. Server constructs feature vectors combining cuisine type, ingredient set, calorie level, spice level, and user preference scores. Server trains a classification or ranking network on positive and negative labels using a loss function optimized for ranking, such as a pairwise or listwise ranking loss. Server then uses this model to re-rank or filter outputs of the generative AI model, thereby increasing acceptance rates without re-training the generative model itself. This improves computational efficiency by avoiding repeated generation of clearly suboptimal suggestions.

In another embodiment, server fine-tunes a generative model or adjusts prompt templates based on feedback statistics. For example, if the user repeatedly rejects spicy dishes, server modifies the prompt sentence to include stronger constraints such as “avoid spicy dishes.” If the user consistently accepts certain cuisines, server modifies the prompt to emphasize those cuisines. Because server makes these changes automatically based on quantitative feedback, the system continuously adapts and reduces the need for manual configuration. This dynamic prompt optimization improves the technical functioning of the system by reducing the number of low-quality generations and the amount of data that must be transmitted and rendered.

Server also implements a feedback loop that uses intake history and time-series user information to predict future health states and proactively adjust prompts. Server aggregates past meal proposals that were accepted or ordered, builds a chronological sequence of nutrient vectors, and aligns them with weight measurements, blood pressure readings, or other health indicators. Server trains a time-series prediction model, such as a recurrent neural network, on these sequences. Server uses a loss function such as mean absolute error to train the model to forecast future weight or other metrics. When generating new prompts, server consults this model to determine energy targets or nutrient constraints likely to steer the user toward desired health outcomes. For instance, if forecasts indicate a risk of continued weight gain, server inserts stricter caloric constraints in future prompt sentences.

This architecture provides multiple technical effects. By unifying heterogeneous user data into compact feature vectors and standardized data structures, server reduces redundant computation and accelerates model inference. By encoding constraints and user context in prompt sentences, server allows the generative AI model to produce more relevant outputs directly, which reduces the need for heavy downstream filtering and recalculation. By coupling generative output with a numeric prediction pipeline, server uses predictive computation to refine constraints and to provide more accurate feedback to the user. By maintaining a structured feedback loop, server continuously improves model performance, reduces error rates in predicted acceptance and health impact, and lowers communication and processing costs associated with irrelevant recommendations.

In contrast to manual or rule-based approaches, server and terminal cooperate to execute specific algorithms and dataflows that are not conventional in human decision-making. The emotion classifier uses high-dimensional embeddings and learned attention weights, and the generative model uses transformer layers with multi-head attention, layer normalization, and learned positional encodings. The predictive models use time-series architectures trained with explicit loss functions and gradient-based updates. The prompt generation process translates numeric feature values and constraints into structured natural language with explicit condition clauses, rather than simple descriptive prose. These techniques, and the interaction among them, implement a non-conventional data processing pipeline that improves the operation of the computing system itself-enhancing accuracy, reducing latency, and optimizing resource usage in the generation of personalized meal proposal information. In another embodiment, server integrates with an external supply service through an application programming interface. Server maps generated meal descriptions to available items in a remote menu database, using similarity matching algorithms and optional pre-computed embeddings. When the user accepts a proposal and chooses to place an order, server sends an order request to the external service. This integration demonstrates concrete use in the physical world by linking computed recommendations to actual delivery and consumption of meals. The system can also be deployed on edge computing hardware co-located with health monitoring devices, where reduced network round-trips and more efficient data encoding contribute to lower power consumption and improved responsiveness. Different variations of the embodiments are possible. Server may use different neural architectures, such as convolutional networks for image-based emotion detection or graph-based networks for ingredient compatibility modeling. Terminal may be implemented as a vehicle-mounted device, a home appliance interface, or a kiosk. The generative model may be trained or fine-tuned using domain-specific corpora, and constraint encoding in the prompt sentence may be adapted to different dietary guidelines or regulatory requirements. The essential feature is that server systematically transforms heterogeneous, time-varying user information into structured feature values, generates a machine-optimized prompt sentence embedding constraints, invokes a generative AI model in a controlled manner, performs numeric prediction of health impact, and closes a feedback loop to adapt internal models and prompts based on structured user reactions, thereby realizing a concrete improvement in computer-implemented generation and management of personalized meal proposal information.

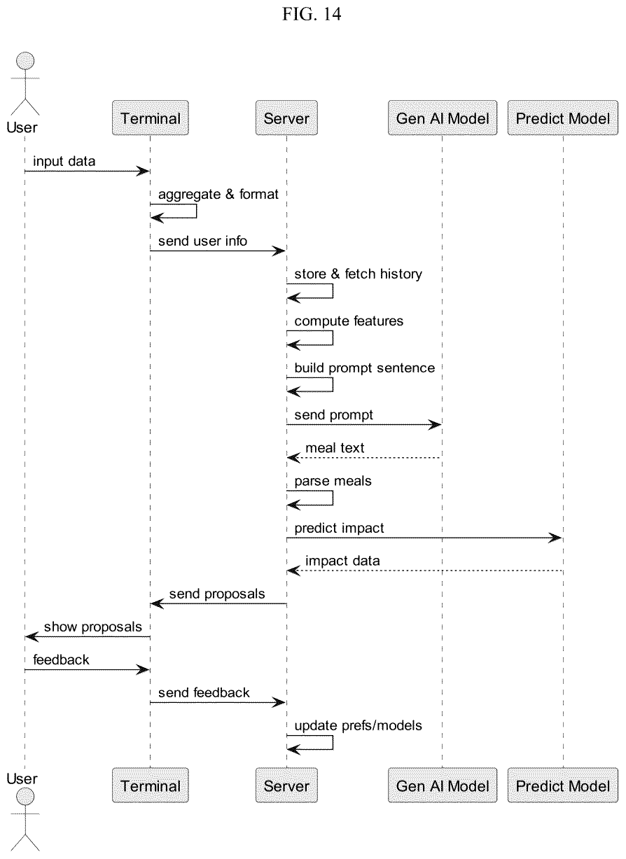

The following describes the processing flow using FIG. 14.

### Step 1

User operates the terminal to input user information.

User enters physical information (age, height, weight, sex), lifestyle information (exercise frequency, typical bedtime, work style), preference information (liked cuisines, disliked ingredients, dietary restrictions), and emotional information (current mood in free text such as “I feel stressed today”) via graphical input fields, selection boxes, and optional voice input. Input: Raw user entries (keystrokes, taps, voice) and sensor values from connected devices.

Output: Structured in-memory records on the terminal representing physical, lifestyle, preference, and emotional information with timestamps.

Terminal converts raw inputs into internal data structures, normalizes units (e.g., converts weight to kilograms, time to 24-hour format), and attaches a user identifier and current timestamp to each field.

### Step 2

Terminal acquires sensor-based lifestyle data and aggregates it.

Terminal reads heart rate, step counts, and sleep duration from wearable devices via short-range communication and from local health applications.

Input: Raw sensor streams and health app logs (e.g., per-minute steps, per-second heart rate).

Output: Aggregated daily metrics (e.g., total steps, average heart rate, total sleep hours) stored as a lifestyle information record.

Terminal sums values over a day, computes averages, discards corrupted readings, and merges the result into the structured user information prepared in Step 1.

### Step 3

Terminal standardizes user information and transmits it to the server.

Terminal packs physical, lifestyle, preference, and emotional information into a standardized digital data format (e.g., a hierarchical key-value structure) and sends it via a secure communication protocol to the server.

Input: Internal structured records created in Steps 1 and 2.

Output: A single standardized payload transmitted to the server representing current user information.

Terminal serializes the records, compresses data if necessary, encrypts the payload, and issues a network request to the server's endpoint.

### Step 4

Server receives and stores the standardized payload.

Server listens on a communication interface, accepts the payload from the terminal, validates the structure, and writes entries into a storage device in appropriate logical regions (profile, health, lifestyle, preference, emotion).

Input: Standardized user information payload from the terminal.

Output: Persistent database records associated with a user identifier and timestamps.

Server parses the payload, checks required fields and types, maps each section to database tables or equivalent structures, and executes insert or update operations.

### Step 5

Server constructs time-aligned user histories.

Server retrieves recent physical, lifestyle, preference, and emotion records for the user and aligns them along a time axis.

Input: Multiple database records for the same user across different tables and timestamps.

Output: Time-aligned sequences of measurements and events ready for feature extraction. Server uses join operations keyed by user identifier and time intervals, interpolates missing points (e.g., fills gaps in step counts), and discards out-of-range or inconsistent entries.

### Step 6

Server computes feature values describing user state and tendencies.

Server transforms raw historical and current records into compact numerical and categorical features that summarize health state, lifestyle tendency, preference tendency, and emotional state.

Input: Time-aligned sequences and current records from Step 5.

Output: A feature vector that includes BMI, weight trend, average steps, sleep statistics, cuisine scores, and an emotion label with confidence.

Server calculates body mass index, moving averages and slopes for weight, averages and variances for activity and sleep, and counts of accepted and rejected cuisines to derive normalized preference scores.

### Step 7

Server analyzes emotional information using a neural network classifier.

Server processes textual emotional information and optionally other signals to determine the current emotional state.

Input: Emotional text from the payload and, if available, emotion-related signals linked in the database.

Output: An emotion probability distribution and a selected emotion label appended to the feature vector.

Server tokenizes the text, converts tokens to embeddings, feeds them through a trained neural network (for example, with embedding, recurrent or transformer layers, and a softmax output layer), computes probabilities for emotion categories, and selects the maximum-probability label.

### Step 8

Server generates a constraint-aware prompt sentence for the generative AI model.

Server converts the feature vector into a natural language prompt sentence that encodes user context and explicit constraints on nutrients and energy.

Input: Feature vector from Steps 6 and 7 and rule parameters specifying dietary targets.

Output: A prompt sentence that describes user state, preferences, emotion, context, and constraints.

Server selects a template, inserts numerical values (e.g., “1800-2000 kcal per day”, “low salt”), concatenates context sections, and appends a task description instructing the generative AI model on required output structure.

### Step 9

Server calls the generative AI model with the prompt sentence.

Server sends the prompt sentence to a generative AI model endpoint or local model and requests generation of meal proposal information.

Input: Prompt sentence constructed in Step 8 and generation parameters (maximum length, temperature, etc.).

Output: Natural-language meal proposal information that includes one or more meals, descriptions, and optionally calorie and nutrient estimates.

Server transmits the prompt to the model, waits for the streaming or batched response, and aggregates tokens into complete sentences and sections corresponding to individual meal proposals.

### Step 10

Server extracts nutritional and structural information from the generated text.

Server parses the meal proposal information to identify dishes, ingredients, portions, and nutritional attributes.

Input: Generated natural-language output from the generative AI model.

Output: A structured representation for each proposed meal, including dish name, ingredient list, portion size, and nutritional vectors.

Server applies text parsing and pattern matching, maps ingredient phrases to entries in a nutrition database, sums nutrient values for ingredients, calculates total energy and macronutrients, and stores these results in intermediate data structures.

### Step 11

Server predicts the influence of proposed meals on the user's health state.

Server feeds the nutritional vectors together with current user feature values to a predictive model that estimates changes in health metrics.

Input: Nutritional vectors from Step 10 and feature vector from Step 6.

Output: Quantitative predictions such as expected weight change, impact on fatigue, or other health indicators for each proposal.

Server organizes nutrients and user features into model inputs, runs a trained neural network or regression model (e.g., LSTM or feed-forward network), computes output values such as predicted weight delta or risk scores, and attaches these numeric results to each meal.

### Step 12

Server composes enriched meal proposal information and sends it to the terminal.

Server combines descriptive meal text, structured nutrition data, and predicted health influence into a single response for user presentation.

Input: Original generative output, structured meal representations, and prediction results from

### Step 11

Output: A response payload containing, for each proposal, text description, numerical nutrition data, and predicted health effects.

Server merges these components into a hierarchical structure, serializes it to a transmission format, and sends it via the communication network to the terminal.

### Step 13

Terminal displays the enriched proposals and captures user reactions.

Terminal receives the response payload and renders each meal proposal with relevant details on the display.

Input: Response payload from Step 12.

Output: Visual presentation of meal proposals and internal records of user choices and optional comments.

Terminal displays meal names, calorie values, nutrient highlights, predicted effects on health and mood, and buttons such as “Accept,” “Reject,” or “Order,” and records which controls the user activates along with any typed feedback.

### Step 14

User reviews the proposals and provides acceptance, rejection, or ordering decisions.

User reads the displayed information, considers health impact and preferences, and chooses whether to accept, reject, or order particular proposals.

Input: On-screen information provided by the terminal.

Output: User actions (button presses or selections) and optional textual feedback indicating satisfaction or reasons for rejection.

User taps appropriate buttons and may enter brief comments like “too spicy” or “more vegetables needed,” which the terminal logs as reaction information.

### Step 15

Terminal sends structured reaction information to the server.

Terminal converts user actions and comments into reaction records and transmits them to the server.

Input: Logged user actions and feedback from Step 14.

Output: Standardized reaction payload identifying each proposal and the corresponding user response.

Terminal associates each reaction with the proposal identifier and timestamp, organizes them into a uniform data structure, and sends them to the server through the communication interface.

### Step 16

Server records reactions as positive and negative example data.

Server receives reaction records and updates storage regions for feedback and preferences.

Input: Reaction payloads from Step 15.

Output: Persistent positive example data (accepted proposals) and negative example data (rejected proposals) linked to user state and prompts.

Server writes each reaction with references to the original prompt sentence, generated text, meal structure, and user feature vector at the time of generation, and flags each reaction as positive or negative for later training.

### Step 17

Server updates preference tendencies and auxiliary recommendation models.

Server processes accumulated feedback to refine estimates of what types of meals the user tends to accept or reject.

Input: Feedback records stored in Step 16 and existing preference statistics.

Output: Updated preference scores and, optionally, updated parameters of a ranking or classification model.

Server increments acceptance and rejection counts per cuisine and attribute (e.g., “spicy,” “high carb”), recalculates normalized preference scores, and may retrain a lightweight model that predicts acceptance probability based on meal attributes and current user state.

### Step 18

Server adapts prompt generation behavior for future interactions.

Server adjusts how future prompt sentences are composed, based on updated preference tendencies and, optionally, outputs of auxiliary models.

Input: Updated preference scores and model outputs from Step 17.

Output: Modified prompt generation rules or parameters that influence subsequent prompt sentences.

Server alters templates by explicitly adding or strengthening language such as “avoid spicy dishes” or “prioritize Japanese-style meals,” adjusts constraint thresholds (e.g., tighter calorie caps), and can change the structure of the task description to request more or fewer options depending on past acceptance patterns.

### Step 19

Server optionally updates or fine-tunes the generative AI and predictive models.

Server uses accumulated positive and negative example data to improve internal models when scheduled or when sufficient new data exists.

Input: Labeled examples containing prompts, generated outputs, user state, and reaction labels.

Output: Updated model weights and configurations that better align outputs with user behavior and health outcomes.

Server constructs supervised training batches, computes gradients of defined loss functions (e.g., classification loss for acceptance prediction or prediction error for health impact), updates model parameters using an optimization algorithm, and stores new model versions or configuration values for subsequent inference.

### Step 20

Server and terminal repeat the cycle with improved personalization and efficiency.

Server uses updated features, models, and prompt generation strategies in future interactions, while terminal continues to gather and forward new user information and reactions. Input: New user information and previous system state (updated models and rules). Output: Subsequent iterations of prompt sentences, generative outputs, predictions, and personalized proposals that reflect ongoing learning.

Server continuously integrates new records with historical data, applies the refined pipeline to generate more accurate and efficient meal proposal information, and thereby improves computation speed, acceptance accuracy, and overall system performance over time.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

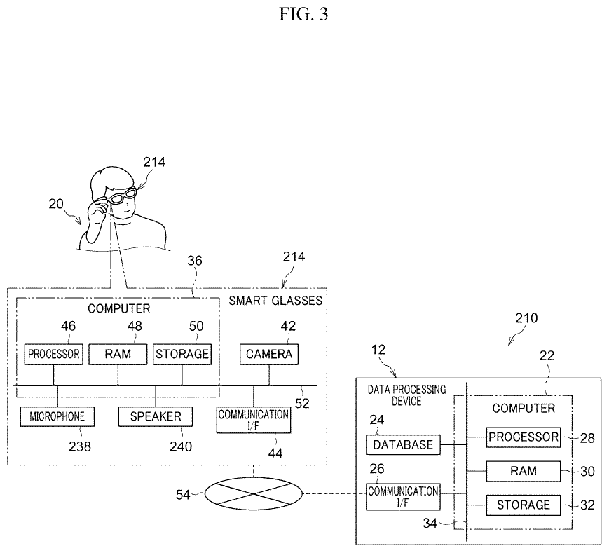

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

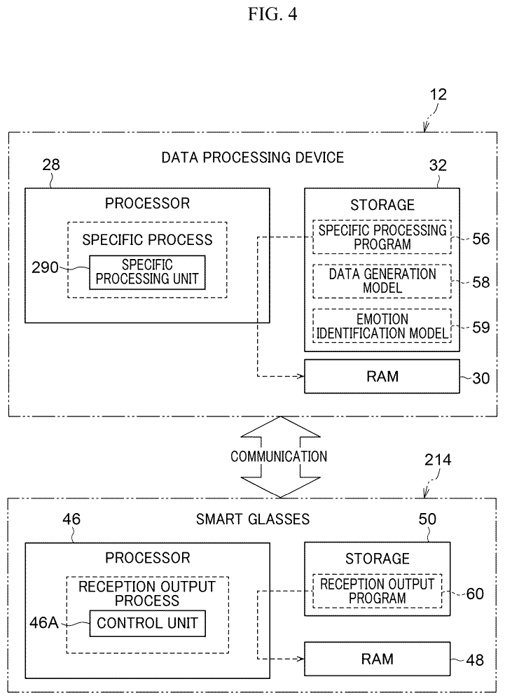

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

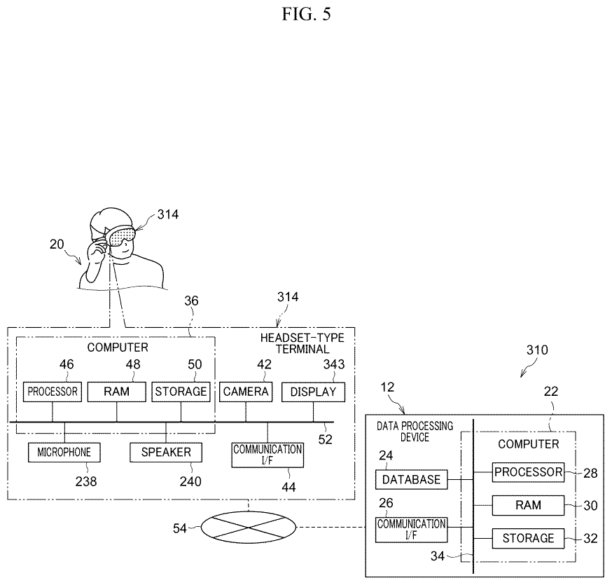

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

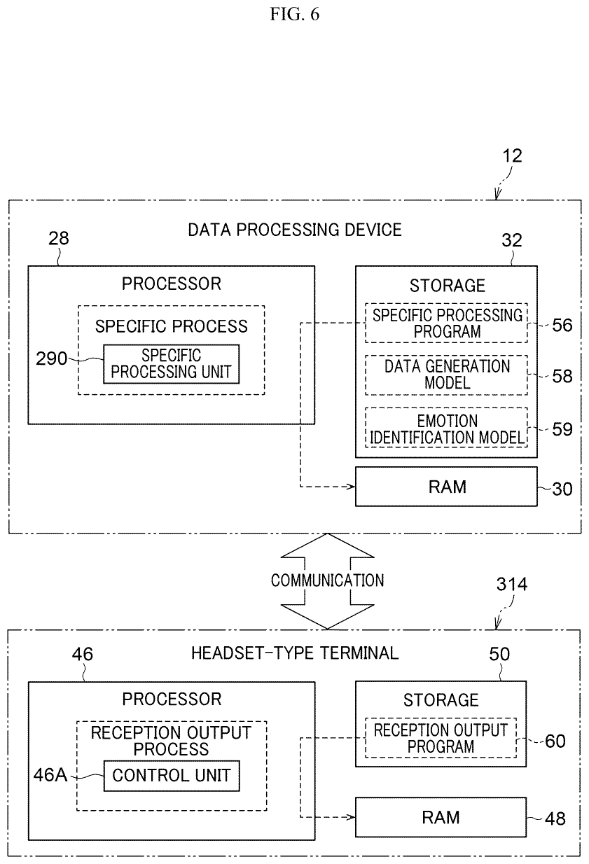

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

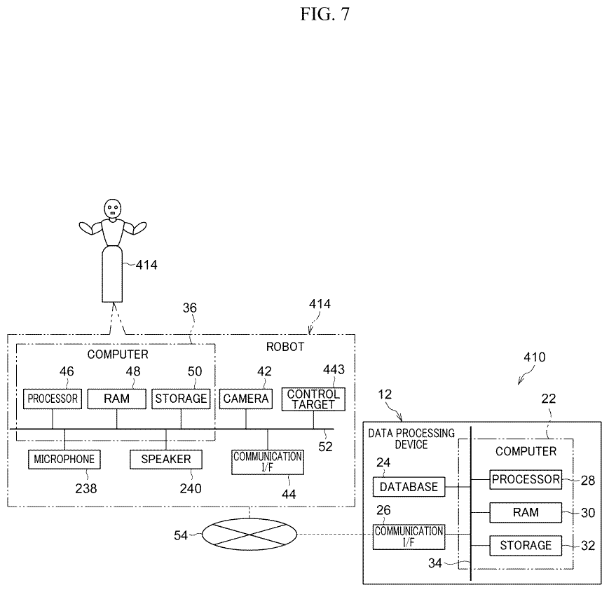

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

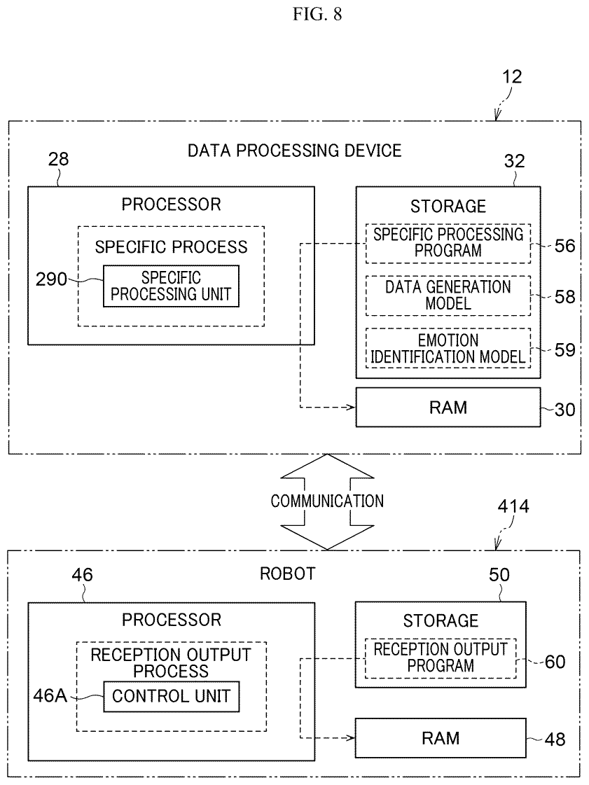

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

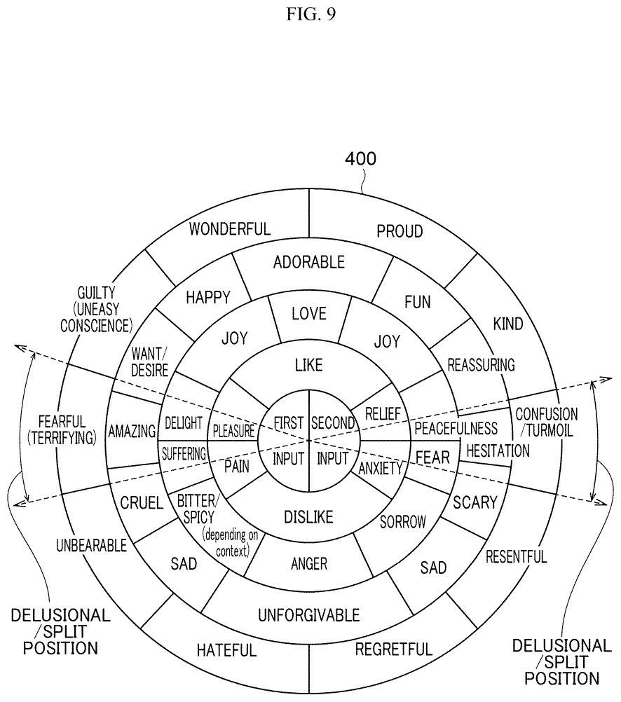

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

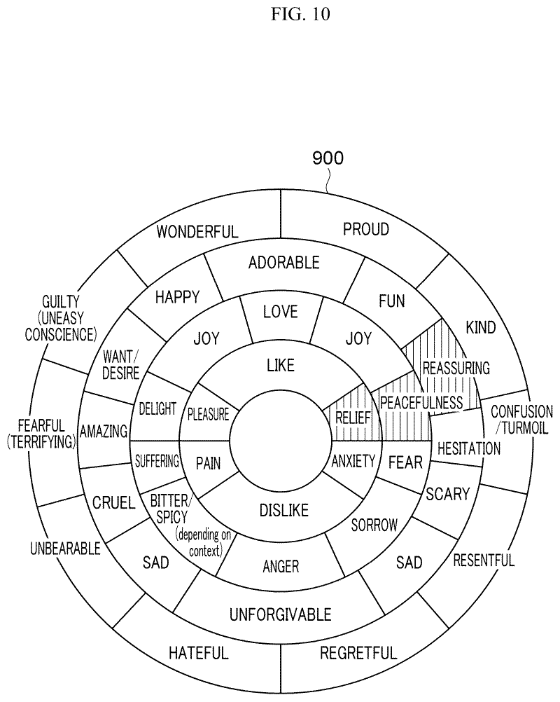

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (Saas).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to
provide an acquisition interface for acquiring bodily condition information, lifestyle information, and meal preference information of a user,
convert the acquired bodily condition information, lifestyle information, and meal preference information into digital information, transmit the digital information to an information processing apparatus via a communication unit, and store the digital information in the information processing apparatus,
acquire biophysical activity information and behavioral history information relating to the user from an external measurement apparatus and an external information service in the information processing apparatus, and aggregate the biophysical activity information and the behavioral history information to generate a health state index,
execute natural language processing on text information acquired from the user and on text information acquired from the external information service in the information processing apparatus, and extract feature information relating to meal preferences, prohibitions, and social situations from the text information to generate a preference profile,
convert the health state index and the preference profile into a numerical feature vector in the information processing apparatus, and generate a prompt sentence summarizing the numerical feature vector and constraint conditions of the user,
supply the prompt sentence and the numerical feature vector as input conditions to a generative information processing model in the information processing apparatus, and cause the generative information processing model to generate meal planning information, structure meal planning information output from the generative information processing model in the information processing apparatus, and store, in a storage unit, meal proposal information including date information, intake time information, dish information, and nutrition information,
transmit the meal proposal information to a terminal apparatus of the user, and cause the terminal apparatus to present the meal proposal information to the user via a display unit, receive evaluation information and additional text information from the user regarding the meal proposal information, and transmit the evaluation information and the additional text information to the information processing apparatus,
execute natural language processing and statistical processing on the evaluation information and the additional text information in the information processing apparatus, and update the preference profile and training information for the generative information processing model, and
execute, in the information processing apparatus, a learning process or a fine-tuning process of the generative information processing model periodically or conditionally based on the updated preference profile and the training information so as to improve accuracy of generation of the meal planning information in a subsequent cycle.

### Supplementary 2

The system according to supplementary 1,
wherein the processor is configured to
calculate nutrition evaluation information including a nutrient distribution index, an energy intake index, and an intake time pattern based on the health state index and the preference profile in the information processing apparatus, and include the nutrition evaluation information in the prompt sentence so that the generative information processing model generates meal planning information in which nutritional balance is taken into account.

### Supplementary 3

The system according to supplementary 1,
wherein the processor is configured to
record the health state index, the meal planning information, and the evaluation information along a time axis in the information processing apparatus, construct a state change model for each user based on the record, and dynamically adjust contents of the prompt sentence and the numerical feature vector according to the state change model so as to monitor a health state of the user and form a feedback loop that automatically modifies the meal planning information.

### Application Example 1

### Supplementary 1

A system comprising a processor,
wherein the processor is configured to
receive biometric information and behavioral information of a user from a terminal via a communication network, standardize the biometric information and the behavioral information, and store the standardized biometric information and behavioral information as a user profile in a storage device,
analyze text information including posting information of the user, the text information being obtained via an external information providing device, by using a natural language processing function, extract preference information relating to meals and social situation information from the text information, and store the preference information and the social situation information in association with the user profile in the storage device,
generate a prompt sentence to be input to a generative AI model based on the user profile, the preference information, and the social situation information, supply the prompt sentence to the generative AI model, and cause the generative AI model to generate a meal plan adapted to a health condition and a living situation of the user and reflecting the preference information and the social situation information,
output the generated meal plan to a display unit of the terminal and accept selection input of the meal plan by the user via the terminal,
acquire candidate menu information provided from an information providing device of an external delivery service device in order to obtain an actually provided food corresponding to the meal plan selected by the user, generate a prompt sentence for the generative AI model based on the candidate menu information and the meal plan, and, by using the prompt sentence, specify, from among the candidate menu information, the actually provided food that most closely matches the meal plan,
transmit order information including the specified actually provided food to the external delivery service device, acquire progress information relating to a delivery state based on the order information from the external delivery service device, and notify the progress information to the terminal, and
acquire evaluation information from the user regarding the actually provided food, update the preference information based on the evaluation information, generate a new prompt sentence reflecting the updated preference information, and supply the new prompt sentence to the generative AI model as a condition for generation of a subsequent meal plan.

### Supplementary 2

The system according to supplementary 1,
wherein the processor is configured to
include, in the prompt sentence, nutrition balance conditions including distribution of nutrients, energy amount, and intake timing based on the biometric information, the behavioral information, the preference information, and the social situation information, and instruct the generative AI model, by the prompt sentence, to generate a plurality of meal plans satisfying the nutrition balance conditions.

### Supplementary 3

The system according to supplementary 1,
wherein the processor is configured to
estimate the health condition of the user by combining the evaluation information, the progress information relating to the delivery state, the biometric information, and the behavioral information, generate a prompt sentence including an adjustment condition for changing contents of the meal plan according to an estimation result, and instruct the generative AI model, by the prompt sentence, to regenerate or modify the meal plan so as to constitute a feedback loop.

### Example 2

### Supplementary 1

A system comprising a processor,
wherein the processor is configured to
acquire user attribute information, physical information, lifestyle information, and preference information by using an input/output apparatus for entering the information, and generate digital data representing the information,
transmit and receive the digital data to and from a storage apparatus via a communication apparatus, and store the digital data in the storage apparatus in association with identification information and time information,
extract the user attribute information, physical information, lifestyle information, and preference information from the stored digital data, generate a prompt sentence including the extracted information and nutritional intake conditions, and cause a generative AI model to generate meal plan data by inputting the prompt sentence to the generative AI model,
analyze nutrient information included in the meal plan data, combine the nutrient information with the stored user physical information and lifestyle information to generate feature data, and input the feature data to a machine learning model to generate prediction result data that predicts future health states and lifestyle changes of the user,
integrate the meal plan data and the prediction result data, convert the integrated information into presentation data for visually presenting the integrated information on a display apparatus of the user, and output the presentation data to the display apparatus, and acquire user satisfaction information and physical condition change information via the display apparatus as feedback data, store the feedback data in the storage apparatus, and update generation conditions of the prompt sentence and parameters of the machine learning model based on the feedback data and the stored digital data.

### Supplementary 2

The system according to supplementary 1,
wherein the processor is configured to
generate the prompt sentence by adding constraint conditions including nutrient balance, energy intake amount, and intake restriction conditions to the prompt sentence based on the stored user attribute information, physical information, lifestyle information, and preference information, and instruct the generative AI model, via the prompt sentence, to generate the meal plan data that satisfies the constraint conditions.

### Supplementary 3

The system according to supplementary 1,
wherein the processor is configured to
calculate a degree of deviation between the prediction result data output from the machine learning model and an actual transition of the health state and lifestyle of the user based on the feedback data and the prediction result data, and, when the degree of deviation satisfies a predetermined condition, automatically reset learning processing of the machine learning model and generation logic of the prompt sentence so as to improve the meal plan data and the prediction result data over time.

### Application Example 2

### Supplementary 1

A system comprising a processor,
wherein the processor is configured to
acquire, via a user information input/output apparatus, user information including physical information, lifestyle information, preference information, and emotional information, convert the acquired user information into a standardized digital data format, and transmit the standardized digital data via a communication network,
receive, via the communication network, the transmitted digital data, store the digital data in an information storage region of a storage device, and manage the stored digital data in association with each user in chronological order,
calculate, on the basis of the stored physical information, lifestyle information, preference information, and emotional information, feature values representing a health state, a lifestyle tendency, a preference tendency, and an emotional state of the user, and generate a prompt sentence in a natural language by using the feature values,
input the prompt sentence to a generative artificial intelligence model and cause the generative artificial intelligence model to generate, in the natural language, meal proposal information that takes into account the health state, the lifestyle tendency, the preference tendency, and the emotional state of the user,
extract nutritional information and intake amount information from the meal proposal information output from the generative artificial intelligence model, combine the nutritional information and the intake amount information with the physical information and the lifestyle information, and numerically predict an influence of the meal proposal information on the health state of the user,
present the meal proposal information output from the generative artificial intelligence model and a prediction result of the influence to a display device corresponding to the user information input/output apparatus, and acquire, from the user, reaction information indicating acceptance or rejection with respect to the meal proposal information, and register the reaction information in the storage device as positive example data and negative example data, and update an operational characteristic of at least one of the generative artificial intelligence model and a prompt sentence generation process by using the positive example data and the negative example data so as to train the system to adapt contents of generation of subsequent meal proposal information to the preference tendency and the emotional state of the user.

### Supplementary 2

The system according to supplementary 1,
wherein the processor is configured to
embed, into the prompt sentence, constraint conditions including amounts of nutrients, an amount of energy, and intake restriction conditions on the basis of the physical information, the lifestyle information, and the preference information of the user, and instruct the generative artificial intelligence model to generate meal plan information that satisfies the constraint conditions so as to generate nutritionally balanced meal plan information.

### Supplementary 3

The system according to supplementary 1,
wherein the processor is configured to
store, in the storage device, intake history based on the meal proposal information and time-series information including the physical information, the lifestyle information, and the emotional information of the user, predict, on the basis of the intake history and the time-series information, a future change in at least one of a health state and a lifestyle tendency of the user, and generate a new prompt sentence reflecting a prediction result and input the new prompt sentence to the generative artificial intelligence model so as to configure a feedback loop that automatically adjusts the meal proposal information.

What is claimed is:

1. A system comprising:
circuitry configured to:
receive, via a communication interface coupled to a packet-switched network, bodily condition information, lifestyle information, and preference information from a terminal device, convert the received information into digital data, and store the digital data in a storage device;
acquire biophysical measurement data from a sensor device via the communication interface, tokenize a prompt sentence incorporating the stored digital data and the biophysical measurement data, transmit the tokenized prompt sentence to an inference engine executing a generative AI model, and decode an output token sequence from the inference engine to obtain a resource utilization plan as a text string; and
parse the text string to extract structured plan data, and transmit the structured plan data to a display device of the terminal device via the communication interface.

2. The system according to claim 1, wherein the circuitry is configured to provide an acquisition interface for receiving bodily condition information, lifestyle information, and preference information from the terminal device, convert the received information into digital data, and store the digital data in a storage device.

3. The system according to claim 2, wherein the circuitry is configured to acquire biophysical measurement data from a sensor device coupled to the terminal device via the communication interface, and store the biophysical measurement data in the storage device in association with a user identifier.

4. The system according to claim 3, wherein the circuitry is configured to generate the prompt sentence incorporating the stored digital data and the biophysical measurement data, and configure inference parameters including temperature and maximum output length prior to transmission to the inference engine.

5. The system according to claim 4, wherein the circuitry is configured to generate a prompt sentence instructing the generative AI model to generate a resource utilization plan that accounts for attribute balance based on the bodily condition information and the preference information.

6. The system according to claim 5, wherein the circuitry is configured to adjust the resource utilization plan based on changes in the biophysical measurement data received over time, and retransmit the tokenized prompt sentence to the inference engine when a detected change in the biophysical measurement data exceeds a threshold.

7. The system according to claim 1, wherein the circuitry is configured to receive feedback from the terminal device indicating whether the resource utilization plan was accepted or modified by the user, incorporate the feedback into a preference model stored in the storage device, and update the prompt sentence for subsequent plan generation.

8. The system according to claim 7, wherein the circuitry is configured to update the preference model based on accumulated feedback data, and use the updated preference model to improve alignment between generated resource utilization plans and user preferences in subsequent sessions.

9. The system according to claim 1, wherein the circuitry is configured to receive health indicator data from the terminal device or a connected sensor via the communication interface, and incorporate the health indicator data into the prompt sentence to generate a health-aware resource utilization plan.

10. The system according to claim 9, wherein the circuitry is configured to detect deviations in health indicator data from target ranges stored in the storage device, generate a prompt sentence for the generative AI model to adjust the resource utilization plan in response to the detected deviations, and transmit the adjusted plan to the terminal device.

11. The system according to claim 1, wherein the circuitry is configured to receive from the terminal device a user preference update specifying changes to the preference information, update the stored digital data in the storage device, and regenerate the prompt sentence for the inference engine based on the updated preference information.

12. The system according to claim 11, wherein the circuitry is configured to detect conflicts between updated preference information and constraints derived from the biophysical measurement data, and generate a revised prompt sentence for the inference engine to resolve the conflicts in the generated resource utilization plan.

13. The system according to claim 1, wherein the circuitry is configured to generate a plurality of candidate resource utilization plans by varying inference parameters in successive transmissions to the inference engine, evaluate the candidates based on attribute balance scores computed from the biophysical measurement data, and select a plan satisfying a balance threshold.

14. The system according to claim 13, wherein the circuitry is configured to transmit the selected resource utilization plan to the terminal device, receive a user selection of one of the candidate plans, and store the selected plan in the storage device in association with the user identifier.

15. The system according to claim 1, wherein the circuitry is configured to receive schedule information from the terminal device via the communication interface, and incorporate the schedule information into the prompt sentence to generate a resource utilization plan adapted to the user's schedule.

16. The system according to claim 15, wherein the circuitry is configured to detect schedule conflicts based on the schedule information and the generated resource utilization plan, and generate a revised prompt sentence for the inference engine to resolve the conflicts.

17. The system according to claim 1, wherein the circuitry is configured to monitor changes in the biophysical measurement data received from the sensor device over time, maintain a time-series record of biophysical measurement data in the storage device, and generate prompt sentences that incorporate the time-series record for longitudinal plan adjustment.

18. A system comprising:
circuitry configured to:
receive, via a communication interface coupled to a packet-switched network, bodily condition information, lifestyle information, and preference information from a terminal device, convert the received information into digital data, and store the digital data in a storage device;
acquire biophysical measurement data from a sensor device via the communication interface, tokenize a prompt sentence incorporating the stored digital data and the biophysical measurement data, transmit the tokenized prompt sentence to an inference engine executing a generative AI model, and decode an output token sequence to obtain a resource utilization plan as a text string;
parse the text string to extract structured plan data, and adjust the structured plan data based on feedback received from the terminal device and changes in the biophysical measurement data; and
transmit the adjusted structured plan data to a display device of the terminal device via the communication interface.

19. The system according to claim 18, wherein the circuitry is configured to generate a plurality of candidate resource utilization plans by varying inference parameters in successive transmissions to the inference engine, evaluate the candidates based on attribute balance scores computed from the biophysical measurement data, select a plan satisfying a balance threshold, and transmit the selected plan to the terminal device via the communication interface.

20. A method comprising:
receiving, via a communication interface coupled to a packet-switched network, bodily condition information, lifestyle information, and preference information from a terminal device, converting the received information into digital data, and storing the digital data in a storage device;
acquiring biophysical measurement data from a sensor device via the communication interface, tokenizing a prompt sentence incorporating the stored digital data and the biophysical measurement data, transmitting the tokenized prompt sentence to an inference engine executing a generative AI model, and decoding an output token sequence from the inference engine to obtain a resource utilization plan as a text string; and
parsing the text string to extract structured plan data, and transmitting the structured plan data to a display device of the terminal device via the communication interface.
